(19) United States
(12) Patent Application Publication
(10) Pub. No.: US 2013/0123367 A1
(43) Pub. Date: May 16, 2013

(54) VINYLOGOUS CHALCONE DERIVATIVES AND THEIR MEDICAL USE

(71) Applicants: Thomas Erker, Vienna (AT); Gerda Brunhofer, Vienna (AT); Ulrich Jäger, Vienna (AT); Katrina Vanura, Vienna (AT); Verena Dirsch, Vienna (AT); Elke Heiss, Vienna (AT)

(72) Inventors: Thomas Erker, Vienna (AT); Gerda Brunhofer, Vienna (AT); Ulrich Jäger, Vienna (AT); Katrina Vanura, Vienna (AT); Verena Dirsch, Vienna (AT); Elke Heiss, Vienna (AT)

(73) Assignee: MEDIZINISCHE UNIVERSITAT WIEN, Vienna (AT)

(21) Appl. No.: 13/811,739

(22) PCT Filed: Jul. 27, 2011
(86) PCT No.: PCT/EP11/62945

§ 371 Date: Jan. 23, 2013

(30) Foreign Application Priority Data
Jul. 28, 2010 (EP) 10171061.4

(51) Int. Cl. C07C 49/255; C07C 49/577; C07C 323/22

(52) U.S. Cl. CPC C07C 49/255; C07C 323/22; C07C 49/577
USPC 514/682; 568/331; 514/685; 568/43; 568/328; 568/327; 514/681

(57) ABSTRACT

The present invention relates to vinylogous chalcone derivatives, in particular the compounds of formula (I) as described and defined herein, pharmaceutical compositions comprising these compounds, and their medical use, including their use in the treatment or prevention of cancer, in particular malignant hematological diseases/disorders.

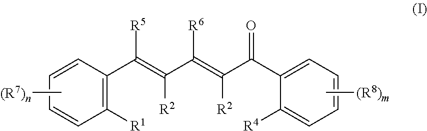

The present invention relates to vinylogous chalcone derivatives, in particular the compounds of formula (I), (II), (III) or (IV) as described and defined herein, pharmaceutical compositions comprising these compounds, and their medical use, including their use in the treatment or prevention of cancer, in particular malignant hematological diseases/disorders.

Malignant hematological diseases are a heterogeneous group of myeloid or lymphoid clonal proliferations characterized by morphological, immunological features and genetics (Swerdlow S H, et al. WHO Classification of Tumours of Haematopoietic and Lymphoid Tissues. IARC Press Lyon 2008). The clinical course can be chronic and indolent or acute. In general, acute diseases (leukemias and aggressive lymphomas) can be cured in approximately half of the patients, while the other patients die from their disease. Of course, variations in subentries with higher or lower cure rates do exist. Chronic leukemias and indolent lymphomas can be well controlled for years in most cases and sometimes do not even require therapy. However, the cure rate of these patients is low and the course of the disease is characterized by frequent recurrence.

Current treatment options include conventional chemotherapy (alkylating agents, anthracyclines, steroids, etc.), small molecules (e.g. kinase inhibitors), and immunotherapy (autologous and allergenic stem cell transplantation).

For most diseases combination or sequential therapy is required to achieve optimal results. Low cure rates and frequent relapses show that novel agents for mono or combination therapies have still to be explored.

In recent years, many advances have been made in the development of anti-cancer drugs, the forms lying on small molecules interfering with aberrant, cancer-specific proteins. An example for these small proteins is the BH3 mimetic ABT-737 which mimics the physiological antagonists of BCL-2 and BCLX_{L} showing promising anti-leukemic action in particular when used in co-treatment with other cytotoxic agents (Mason, K D et al. The BH3 mimetic compound, ABT-737, synergizes with a range of cytotoxic chemotherapy agents in chronic lymphocytic leukemia. Leukemia 2009, aheadofprint). Other approaches try to assess the usefulness of naturally occurring compounds for cancer treatment, alone or in combination with established drugs. Widely known is the group of flavonoids (resveratrol & derivates, flavopiridol) which have been shown to display cytotoxic activity in a variety of cancer cell lines and patient primary cells, including leukemic cells, and which have been developed and are being tested in phase I and II clinical studies, respectively, and show activity in chronic lymphocytic leukemia (CLL), particularly in patients with deletion 11q and 17p (Billard, C et al. Leukemia and Lymphoma 2002, 43, 1991-2002; Byrd, J C et al. Blood 2007, 109, (2), 399-404; Estrov, Z et al. Blood 2003, 102, 987-995; Hauswirth, A W et al. Haematologica 2008, 93, (1), 14-19; Lin, T S et al. Journal of Clinical Oncology: Official Journal of the American Society of Clinical Oncology 2010, 28, (3), 418-423; Lin, T S et al. Journal of Clinical Oncology Official Journal of the American Society of Clinical Oncology 2009, 27, (35), 6012-6018; Park, J W et al. Cancer Letters 2001, 163, 43-49; Phelps, M A et al. Blood 2009, 113, 2637-2645; Tsan, M F et al. Leukemia and Lymphoma 2002, 43, 983-987; Wieder, T et al. Leukemia 2001, 15, 1735-1742).

Flavopiridol (alvocidib) is a synthetic flavone derivative and acts as potent growth inhibitor of diverse human tumor cell lines by induction of apoptosis also including hematopoietic cell lines (Karp, J E et al. Clinical Cancer Research 2005, 11, (23), 8403-8412). Lin reviews that in phase ½ studies flavopiridol showed an increased binding to human plasma proteins resulting in inadequate in vivo plasma drug concentration (Lin, T. Current Hematologic Malignancy Reports 2010, 5, (1), 29-34). But this fact was overcome by a 30-minute intravenous bolus followed by a 4-hour continuous intravenous infusion. Unfortunately, the dose of flavopiridol is limited by life-threatening tumor lysis syndrome.

Another group of compounds exhibiting promising cytotoxic activity are chalcones. Chemically, chalcones are biosynthetic precursors of flavonoids and both natural as well as synthetic derivatives have shown biologic activity in cancer cells (Henmi, K et al. Biological & Pharmaceutical Bulletin 2009, 32, (6), 1109-1113; Kong, Y et al. Bioorganic & Medicinal Chemistry 2010, 18, (2), 971-977; Navarini, A L F et al. European Journal of Medicinal Chemistry 2009, 44, (4), 1630-1637; Yang, X et al. Bioorganic & Medicinal Chemistry Letters 2009, 19, (15), 4385-4388; Szliszka, E et al. International Journal of Molecular Sciences 2009, 11, (1), 1-13; Pilatova, M et al. In vitro antiproliferative and antiangiogenic effects of synthetic chalcone analogues. Toxicology in Vitro 2010, In Press, Uncorrected Proof; Cuendet, M et al. Cancer Prevention Research 2010, 3, (2), 221-232). Recent studies show that chalcone derivatives show cytotoxic activity in different tumor cell lines also including hematological malignancies. (E)-α-Benzylthio chalcones are able to inhibit significantly K562 cell proliferation, a BCR-ABL positive chronic myeloid leukemic cell line (Reddy, M V R et al. Bioorganic & Medicinal Chemistry 2010, 18, (6), 2317-2326). The chemical synthesis of chalcones and chalcone derivatives is described, e.g., in Kiss, A, Synthese von Chalkonen mit potenzieller COX-II-Selektivitat, Diplomarbeit Universität Wien, 2004.

There is a strong demand for alternative and/or improved means and methods for the medical intervention of cancer, in particular malignant hematological diseases/disorders.

A problem underlying the present invention is thus the provision of alternative and/or improved means and methods for the treatment or prevention of cancer, in particular malignant hematological diseases/disorders. The solution to this technical problem is achieved by providing the embodiments characterized herein below and in the claims.

Accordingly, the present invention relates to a compound of the following formula (I)

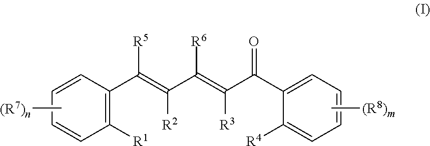

or a pharmaceutically acceptable salt, solvate or prodrug thereof for use in the treatment or prevention of cancer, in particular of a malignant hematological disease/disorder.

R¹ and R² are each independently selected from hydrogen, halogen, —NO₂, —CF₃, —ON, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —ON, O₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl).

Alternatively, R¹ and R² are mutually linked to form a group —C(R⁹)═C(R⁹)—.

R³ and R⁴ are each independently selected from hydrogen, halogen, —NO₂, —CF₃, —CN, C₁₋₄ alkyl, —OH, —O(C alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ e, alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl).

Alternatively, R³ and R⁴ are mutually linked to form a group —CH(R⁹)— or —CH(R⁹)—CH(R⁹)—.

R⁵ and R⁶ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), or phenyl optionally substituted with one or more groups independently selected from halogen, —NO₂, —CF₃, —CN, O₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl).

Each R⁷ is independently selected from halogen, —NO₂, —CF₃, —CN, O₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(O₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). If n is equal to or greater than 2, two groups R⁷ which are attached to adjacent carbon atoms may also be mutually linked to form a group —C(R⁹)═C(R⁹)—C(R⁹)═C(R⁹)— or a group —O—CH(R⁹)—O—.

Each R⁸ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). If m is equal to or greater than 2, two groups R⁸ which are attached to adjacent carbon atoms may also be mutually linked to form a group —C(R⁹)═C(R⁹)—C(R⁹)═C(R⁹)— or a group —O—CH(R⁹)—O—.

Each R⁹ is independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ d alkyl)(C₁₋₄ alkyl), —SO₂—NH₂, —SO₂—NH(C₁₋₄ alkyl), or —SO₂—N(C₁₋₄ alkyl)(C₁₋₄ alkyl).

n is 0, 1, 2, 3, or 4.
m is 0, 1, 2, 3, or 4.

The present invention also relates to a pharmaceutical composition comprising a compound of formula (I), (II), (III) or (IV), as described and defined herein, or a pharmaceutically acceptable salt, solvate or prodrug thereof, and a pharmaceutically acceptable excipient, for use in the treatment or prevention of cancer, in particular of a malignant hematological disease/disorder.

Moreover, the present invention relates to a method of treating or preventing cancer, in particular a malignant hematological disease/disorder, the method comprising the administration of a compound of formula (I), (II), (III) or (IV), as described and defined herein, or a pharmaceutically acceptable salt, solvate or prodrug thereof, or a pharmaceutical composition comprising any of the aforementioned entities and a pharmaceutically acceptable excipient, to a subject (preferably, a human) in need of such a treatment or prevention.

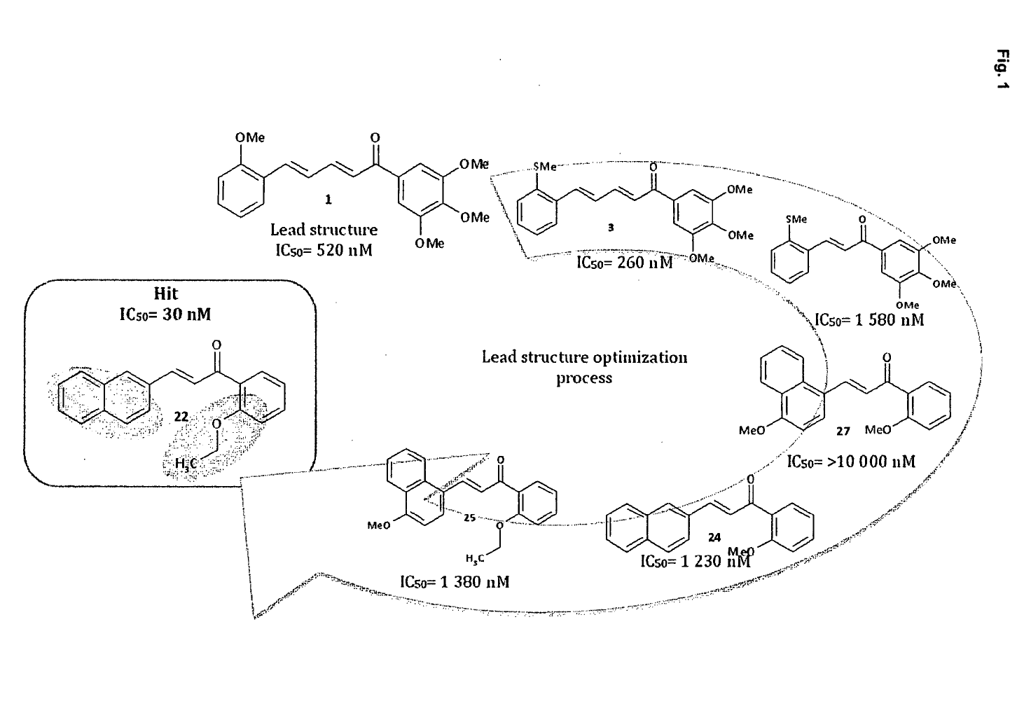

The present invention provides highly active synthetic vinylogous chalcones derived from the chalcone scaffold, in particular the compounds Of formula (I), (II), (III) or (IV). Cytotoxic compounds were found and developed by modifying the chalcone lead structure concurrent with in vitro tests to evaluate their cytotoxic potential in different cancer and non-cancer cell lines. Experimental data were fed back into the chemical model to further optimize the structure of the molecules thus attaining derivatives with particularly high activity. It was surprisingly found that the length of the spacer connecting the two phenyl rings of the general chalcone scaffold was very important for cytotoxicity. Extension of the spacer by one more double bond increased anti-proliferative activity. The medium throughput screening on cancer and non-cancer cell lines was the starting point of the design of a structure-activity relationship (SAR) guided library of new compounds with focus on cytotoxic activity. In a first round, the compounds were tested on colon cancer (SW480) as well as on melanoma cell lines (518A2), modified, and again tested in vitro. Starting from compound 1 which was found to be highly cytotoxic, different modifications on the aldehyde as well as the acetophenone moiety of the molecule were introduced. Thus, information about structural requirements that increased or decreased cytotoxic activity was obtained. It was found that a planar lipophilic part on one side and an ortho-alkoxy group on the other side of the molecule increases cytotoxic activity. Based on the results obtained by this feedback loop, compounds with IC₅₀ values below 1 μM were found. Furthermore, especially compounds having one additional double bond in the spacer showed high cytotoxic activity. The lead structure optimization process, in which compound 22 was developed, is shown in FIG. 1. The compounds were thoroughly tested regarding their efficacy in inhibiting proliferation and viability in cancer cell lines of the hematopoietic system. From these tests, compounds according to the invention emerged showing high cytotoxicity at low concentrations thus displaying promising potential as novel anti-cancer drugs. Accordingly, the compounds of the present invention, in particular the compounds of formula (I), (II), (III) or (IV), are useful in the treatment or prevention of cancer and, in particular, in the treatment or prevention of malignant hematological diseases/disorders.

The cancer to be treated or prevented with the compounds or the pharmaceutical compositions according to the present invention is preferably a non-solid cancer and, more preferably, a malignant hematological disease/disorder, such as myeloproliferative disorders (MPD), including, e.g., acute myeloid leukaemia (AML), chronic myeloid leukaemia (CML), or chronic BCR-ABL negative MPDs, or a lymphoid cancer, which can be a B-cell malignancy, such as B-cell lymphoma, non-Hodgkin lymphoma or B-cell derived chronic lymphatic leukemia (B-CLL), or a T-cell malignancy, such as, e.g., T-cell acute lymphoblastic leukaemia (T-ALL). However, solid cancers can also be treated or prevented with the compounds or the pharmaceutical compositions of the invention.

The compounds or the pharmaceutical compositions of the present invention, including the compounds of formula (I), (II), (III) or (IV), are particularly effective and, thus, particularly useful in the medical intervention of a malignant hematological disease/disorder, as also demonstrated in the examples. The malignant hematological disease/disorder to be treated or prevented in accordance with the invention is preferably selected from: Hodgkin's disease; non-Hodgkin's lymphoma, including, e.g., follicular non-Hodgkin's lymphoma or diffuse non-Hodgkin's lymphoma (e.g., Burkitt's tumor); peripheral/cutaneous T-cell lymphoma, including, e.g., mycosis fungoides, Sezary's disease, T-zone lymphoma, lymphoepithelioid lymphoma (e.g., Lennert's lymphoma), or peripheral T-cell lymphoma; lymphosarcoma; a malignant immunoproliferative disease, including, e.g., Waldenstrom's macroglobulinaemia, alpha heavy chain disease, gamma heavy chain disease (e.g., Franklin's disease), or immunoproliferative small intestinal disease (e.g., Mediterranean disease); multiple myeloma, including, e.g., Kahler's disease, or myelomatosis; plasma cell leukaemia; lymphoid leukaemia, including, e.g., acute lymphoblastic leukaemia, chronic lymphocytic leukaemia, subacute lymphocytic leukaemia, prolymphocytic leukaemia, hairy-cell leukaemia (e.g., leukemia reticuloendotheliosis), or adult T-cell leukaemia; myeloid leukaemia, including, e.g., acute myeloid leukaemia, chronic myeloid leukaemia, subacute myeloid leukaemia, myeloid sarcoma (e.g., chloroma, or granulocytic sarcoma), acute promyelocytic leukaemia, or acute myelomonocytic leukaemia; chronic BCR-ABL negative myeloproliferative disorders, including, e.g., polycythaemia vera, essential thrombocythemia, or idiopathic myelofibrosis; monocytic leukaemia; acute erythraemia or erythroleukaemia, including, e.g., acute erythraernic myelosis, or Di Guglielmo's disease; chronic erythraemia, including, e.g., Heilmeyer-Schoner disease; acute megakaryoblastic leukaemia; mast cell leukaemia; acute panmyelosis; acute myelofibrosis; or Letterer-Siwe disease.

The compounds or the pharmaceutical compositions of the invention are furthermore useful in the treatment or prevention of other types of cancer, including, for example, breast (mamma) cancer, genitourinary cancer (such as, e.g., prostate tumor, including a hormone-refractory prostate tumor), lung cancer (such as, e.g., small cell or non-small cell lung tumor), gastrointestinal cancer (such as, e.g., hepatoceliular carcinoma, colorectal tumor, colon cancer or gastric cancer), epidermoid cancer (such as, e.g., epidermoid head and/or neck tumor or mouth tumor), melanoma, ovarian cancer, pancreas cancer, neuroblastoma, head and/or neck cancer, bladder cancer, renal cancer, or brain cancer.

As shown in Example 2, a compound of formula (I) according to the invention (compound 22) was found to have an IC₅₀ value on the melanoma cell line 518A2 of 40 nM, which is particularly remarkable since melanoma cells frequently show resistance against anti-cancer agents. Compared to cisplatin having an IC₅₀ value of about 10 μM, the compound of the invention is thus about 250 times as potent as cisplatin. Accordingly, the compounds of formula (I), and in particular compound 22, are also particularly useful in the treatment or prevention of melanoma.

The compound of formula (I) as defined above is described in more detail in the following.

R¹ and R² are each independently selected from hydrogen, halogen (such as, e.g., —F, —Cl, —Br, or —I), —NO₂, —CF₃, —CN, C₁₋₄ alkyl), —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). R¹ is preferably selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl), more preferably from hydrogen, halogen, —NO₂, —OH, —O(C alkyl), —SH, or —S(C₁₋₄ alkyl), and even more preferably R¹ is hydrogen. R² is preferably selected from hydrogen, halogen, —NO₂, —OH, —O(C alkyl), —SH, or —S(C₁₋₄ alkyl), and more preferably R² is hydrogen. Accordingly, it is particularly preferred that R¹ and R² are each hydrogen.

Alternatively, R¹ and R² are mutually linked to form a group —C(R⁹)═C(R⁹)—, preferably a group —CH═CH—.

R³ and R⁴ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). R³ is preferably selected from hydrogen, halogen, —NO₂, —OH, —O(C₁₋₄ alkyl), —SH, or —S(C₁₋₄ alkyl), and more preferably R³ is hydrogen. R⁴ is preferably selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄₄ alkyl), —SO₂—NH₂, —SO₂—NH(C₁₋₄ alkyl), or —SO₂—N(C₁₋₄ alkyl)(C₁₋₄ alkyl), more preferably from hydrogen, halogen, —NO₂, —OH, —O(C₁₋₄ alkyl), —SH, or —S(C₁₋₄ alkyl), and even more preferably R⁴ is hydrogen. Accordingly, it is particularly preferred that R³ and R⁴ are each hydrogen.

Alternatively, R³ and R⁴ are mutually linked to form a group —CH(R⁹)— or —CH(R⁹)—CH(R⁹)—, preferably a group —CH₂— or —CH₂—CH₂—.

R⁵ and R⁶ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, R⁵ and R⁶ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), or alkyl)(C₁₋₄ alkyl). More preferably, R⁵ and R⁶ are each independently selected from hydrogen, halogen, —NO₂, —OH, —O(C₁₋₄ alkyl), —SH, or —S(C₁₋₄ alkyl). Even more preferably, R⁵ and R⁶ are each hydrogen.

Each R⁷ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, each R⁷ is independently selected from halogen, —NO₂, —CF₃, —CN, O alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). More preferably, each R⁷ is independently selected from halogen, —NO₂, —OH, —O(C₁₋₄ alkyl), —SH, or —S(C₁₋₄ alkyl). Even more preferably, each R⁷ is independently selected from halogen, —OH, —OCH₃, —OCH₂CH₃, —SH, —SCH₃, —SCH₂CH₃. If n is equal to or greater than 2, two groups R⁷ which are attached to adjacent carbon atoms may also be mutually linked to form a group —C(R⁹)═C(R⁹)—C(R⁹)═C(R⁹)— (such as, e.g., a group —CH═CH—CH═CH—) or a group —O—CH(R⁹)—O— (such as, e.g., a group —O—CH₂—O—).

Each R⁸ is independently selected from halogen, —NO₂, —CF₃, —ON, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, each R⁸ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). More preferably, each R⁸ is independently selected from halogen, —NO₂, —OH, —O(C₁—4 alkyl), —SH, or —S(C₁₋₄ alkyl). Even more preferably, each R⁸ is independently selected from halogen, —OH, —OCH₃, —OCH₂CH₃, —SH, —SCH₃, —SCH₂CH₃. If m is equal to or greater than 2, two groups R⁸ which are attached to adjacent carbon atoms may also be mutually linked to form a group —C(R⁹)═C(R⁹)—C(R⁹)═C(R⁹)— (such as, e.g., a group —CH═CH—CH═CH—) or a group —O—CH(R⁹)—O— (such as, e.g., a group —O—CH₂—O—).

Each R⁹ is independently selected from hydrogen, halogen, —NO₂, —CF₃, —CN, O₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, each R⁹ is independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). More preferably, each R⁹ is independently selected from hydrogen, halogen, —NO₂, —OH, —O(C₁₋₄ alkyl), —SH, or —S(C₁₋₄ alkyl). Even more preferably, each R⁹ is independently selected from hydrogen, halogen, —OH, —OCH₃, —OCH₂CH₃, —SH, —SCH₃, —SCH₂CH₃. Most preferably, each R⁹ is hydrogen.

n is 0, 1, 2, 3, or 4. Preferably, n is 0, 1, 2, 3. More preferably, n is 0 or 1. Even more preferably, n is 1.

m is 0, 1, 2, 3, or 4. Preferably, m is 0, 1, 2, 3. More preferably, m is 1 or 3. In a particularly preferred embodiment, m is 1. In a further particularly preferred embodiment, m is 3.

It is to be understood that, if n is 0, the respective phenyl ring (which R⁷ is attached to) is unsubstituted, i.e. is substituted with hydrogen instead of R⁷. Likewise, it is to be understood that, if m is 0, the respective phenyl ring (which R⁸ is attached to) is unsubstituted, i.e. is substituted with hydrogen instead of R⁸.

In a particularly preferred embodiment, m is 1 and R⁸ is —O(C₁₋₄ alkyl), such as —OCH₃, —OCH₂CH₃, —OCH₂CH₂CH₃, or —OCH₂CH₂CH₂CH₃ (preferably —OCH₂CH₃), bound in ortho position to the phenyl ring of the acetophenone moiety of the compound of formula (I), as for example in compound 22. In this embodiment, it is furthermore preferred that R¹ and R² are mutually linked to form a group —C(R⁹)═C(R⁹)—, such as, e.g., —CH═CH—.

In a further preferred embodiment, m is 3 and each R⁸ is —O(C₁₋₄ alkyl), preferably —OCH₃, wherein the three groups R⁸ are bound to the 3,4,5-positions of the phenyl ring of the acetophenone moiety of the compound of formula (I), as for example in compound 1.

A particularly preferred compound of formula (I) is compound 1 or compound 22 as shown in the following:

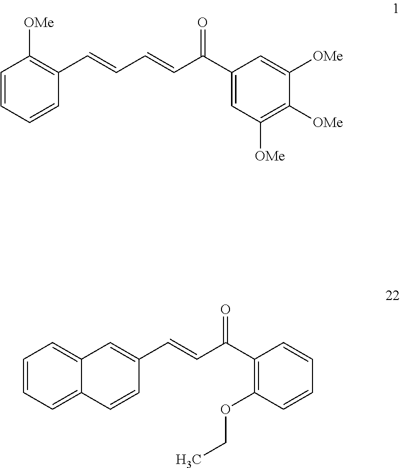 [SMILES: COc1ccccc1/C=C/C=C/C(=O)c1cc(OC)c(OC)c(OC)c1 ; CCOc1ccccc1C(=O)/C=C/c1ccc2ccccc2c1]

or a pharmaceutically acceptable salt, solvate or prodrug thereof. Accordingly, the invention relates to compound 1 or compound 22, as defined above, or a pharmaceutically acceptable salt, solvate or prodrug thereof for use in the treatment or prevention of cancer, in particular of a malignant hematological disease/disorder.

In one embodiment described above, R¹ and R² are mutually linked to form a group —C(R⁹)═C(R⁹)—. Accordingly, the compound of formula (I) as defined above may be a compound of the following formula (II)

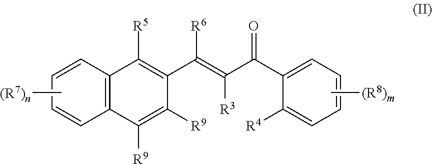

or a pharmaceutically acceptable salt, solvate or prodrug thereof, wherein R³, R⁴, R⁵, R⁶, R⁷, R⁸, R⁹, n, and m have the meanings or the preferred meanings defined herein above for the compound of formula (I). The invention thus relates to a compound of formula (II), as described and defined herein, or a pharmaceutically acceptable salt, solvate or prodrug thereof for use in the treatment or prevention of cancer, in particular of a malignant hematological disease/disorder.

In a further embodiment described above, R³ and R⁴ are mutually linked to form a group —CH(R⁹)—. Accordingly, the compound of formula (I) as defined above may be a compound of the following formula (III)

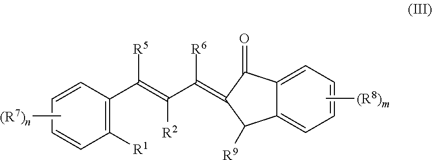

or a pharmaceutically acceptable salt, solvate or prodrug thereof, wherein R¹, R², R⁵, R⁶, R⁷, R⁸, R⁹, n, and m have the meanings or the preferred meanings defined herein above for the compound of formula (I). The invention hence relates to a compound of formula (III), as described and defined herein, or a pharmaceutically acceptable salt, solvate or prodrug thereof for use in the treatment or prevention of cancer, in particular of a malignant hematological disease/disorder.

In yet a further embodiment described above. R³ and R⁴ are mutually linked to form a group —CH(R⁹)—CH(R⁹)—. Accordingly, the compound of formula (I) as defined above may be a compound of the following formula (IV)

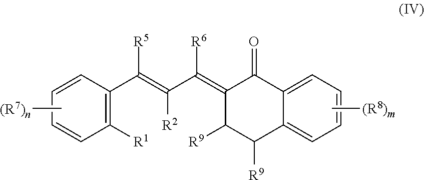

or a pharmaceutically acceptable salt, solvate or prodrug thereof, wherein R¹, R², R⁵, R⁶, R⁷, R⁸, R⁹, n, and m have the meanings or the preferred meanings defined herein above for the compound of formula (I). The invention thus relates to a compound of formula (IV), as described and defined herein, or a pharmaceutically acceptable salt, solvate or prodrug thereof for use in the treatment or prevention of cancer, in particular of a malignant hematological disease/disorder.

The present invention furthermore provides novel compounds. These compounds are described herein and are characterized by the following formula (Ia):

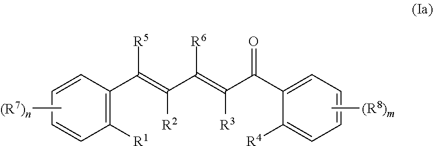

or a pharmaceutically acceptable salt, solvate or prodrug thereof, wherein R¹, R², R³, R⁴, R⁵, R⁶, R⁷, R⁸, R⁹, n and m have the meanings and preferred meanings as described and defined herein above for the compound of formula (I), and further wherein: R¹ and R² are mutually linked to form a croup —C(R⁹)═C(R⁹)—; and/or R³ and R⁴ are mutually linked to form a group —CH(R⁹)— or —CH(R⁹)—CH(R⁹)—.

In one embodiment, the compound of formula (Ia) as defined above is a compound of one of the following formulae (IIa), (IIIa), or (IVa):

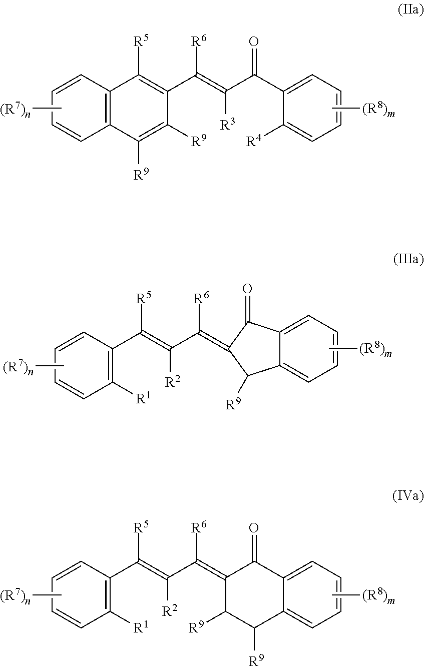

or pharmaceutically salts, solvates or prodrugs thereof, wherein R³, R⁴, R⁵, R⁶, R⁷, R⁸, R⁹, n and m in formula (IIa) as well as R¹, R², R⁵, R⁶, R⁷, R⁸, R⁹, n and m in formulae (IIIa) and (IVa) have the meanings and preferred meanings as described and defined herein above for the compound of formula (I).

Accordingly, the present invention relates to the compounds of formula (Ia), (IIa), (IIIa), or (IVa), as described and defined herein. The compounds of formula (Ia), (IIa), (IIIa), or (IVa), as provided in the context of the present invention, are particularly useful in a medical setting, i.e., as pharmaceuticals. Accordingly, the present invention relates to a compound of formula (Ia), (IIa), (IIIa) or (IVa) or a pharmaceutically acceptable salt, solvate or prodrug thereof for use as a medicament. The invention likewise provides a pharmaceutical composition comprising a compound of formula (Ia), (IIa), (IIIa) or (IVa), as described and defined herein, or a pharmaceutically acceptable salt, solvate or prodrug thereof, and a pharmaceutically acceptable excipient. The invention further relates to a method of treating or preventing a disease or disorder, the method comprising the administration of a compound of formula (Ia), (IIa), (IIIa) or (IVa) or a pharmaceutically acceptable salt, solvate or prodrug thereof, or a pharmaceutical composition comprising any of the aforementioned entities and a pharmaceutically acceptable excipient, to a subject (preferably a human) in need of such a treatment or prevention. As is evident from the disclosure of the present invention and as also demonstrated in the examples, the compounds of formula (Ia), (IIa), (IIIa), or (IVa), as described and defined herein, are particularly useful in the treatment or prevention of cancer, including (but not being limited to) the treatment or prevention of a malignant hematological disease/disorder.

In formula (IIa) shown above, the groups R³, R⁴, R⁵, R⁶, R⁷, R⁸, R⁹ and the variables n and m accordingly have the following meanings and preferred meanings:

R³ and R⁴ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, alkyl), —NH₂, —NH(O₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). R³ is preferably selected from hydrogen, halogen, —NO₂, —OH, —O(C₁₋₄ alkyl), —SH, or —S(C₁₋₄ alkyl), and more preferably R³ is hydrogen. R⁴ is preferably selected from hydrogen, halogen, —NO₂, —CF₃, CN, C₁₋₄ alkyl, —OH, —O(C₁₋₄ alkyl), —SH, —S(C₁₋₄ alkyl), 13 NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl), more preferably from hydrogen, halogen, —NO₂, —OH, —O(C₁₋₄ alkyl), —SH, or —S(C₁₋₄ alkyl), and even more preferably R⁴ is hydrogen. Accordingly, it is particularly preferred that R³ and R⁴ are each hydrogen.

Alternatively, R³ and R⁴ are mutually linked to form a group —CH(R⁹)— or —CH(R⁹)—CH(R⁹)—, preferably a group —CH₂— or —CH₂—CH₂—.

R⁵ and R⁶ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, R⁵ and R⁶ are each independently selected from hydrogen, halogen, —NO₂, —CF₃, —ON, C₁₋₄ alkyl, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). More preferably, R⁵ and R⁶ are each independently selected from hydrogen, halogen, —NO₂, —OH, —O(C alkyl), —SH, or —S(C₁₋₄ alkyl). Even more preferably, R⁵ and R⁶ are each hydrogen.

Each R⁷ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, each R⁷ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or alkyl)(C₁₋₄ alkyl). More preferably, each R⁷ is independently selected from halogen, —NO₂, —OH, —O(C₁₋₄ alkyl), —SH, or —S(C₁₋₄ alkyl). Even more preferably, each R⁷ is independently selected from halogen, —OH, —OCH₃, —OCH₂CH₃, —SH, —SCH₃, —SCH₂CH₃. If n is equal to or greater than 2, two groups R⁷ which are attached to adjacent carbon atoms may also be mutually linked to form a group —C(R⁹)═C(R⁹)—C(R⁹)═C(R⁹)— (such as, e.g., a group —CH═CH—CH═CH—) or a group —O—CH(R⁹)—O— (such as, e.g., a group —O—CH₂—O—).

Each R⁸ is independently selected from halogen, —NO₂, —CF₃, —CN, C₁₋₄ alkyl, —OH, —O(C₁₋₄ alkyl), —SH, alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁—4 alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄—N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, each R⁸ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). More preferably, each R⁸ is independently selected from halogen, —NO₂, —OH, —O(C₁₋₄ alkyl), —SH, or —S(C₁₋₄ alkyl). Even more preferably, each R⁸ is independently selected from halogen, —OH, —OCH₃, —OCH₂CH₃, —SH, —SCH₃, —SCH₂CH₃. If m is equal to or greater than 2, two groups R⁸ which are attached to adjacent carbon atoms may also be mutually linked to form a group —C(R⁹)═C(R⁹)—C(R⁹)═C(R⁹)— (such as, e.g., a group —CH═CH—CH═CH—) or a group —O—CH(R⁹)—O— (such as, e.g., a group —O—CH₂—O—).

Each R⁹ is independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁—4 alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, O₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, each R⁹ is independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). More preferably, each R⁹ is independently selected from hydrogen, halogen, —NO₂, —OH, —O(C₁₋₄ alkyl), —SH, or —S(C₁₋₄ alkyl). Even more preferably, each R⁹ is independently selected from hydrogen, halogen, —OH, —OCH₃, —OCH₂C₁₋₁₃, —SH, —SCH₃, —SCH₂CH₃. Most preferably, each R⁹ is hydrogen.

n is 0, 1, 2, 3, or 4. Preferably, n is 0, 1, 2, 3. More preferably, n is 0 or 1. Even more preferably, n is 1.

m is 0, 1, 2, 3, or 4. Preferably, m is 0, 1, 7, 3. More preferably, m is 1 or 3. in a particularly preferred embodiment, m is 1. In a further particularly preferred embodiment, m is 3.

In a particularly preferred embodiment, m is 1 and R⁸ is —O(C₁₋₄ alkyl), such as —OCH₃, —OCH₂CH₃, —OCH₂CH₂CH₃, or —OCH₂CH₂CH₂CH₃ (preferably —OCH₂CH₃), bound in ortho position to the phenyl ring of the acetophenone moiety of the compound of formula (IIa), as for example in compound 22. In this embodiment, it is furthermore preferred that R⁴ is hydrogen.

In a further preferred embodiment, m is 3 and each R⁸ is —O(C₁₋₄ alkyl), preferably —OCH₃, wherein the three groups R⁸ are bound to the 3,4,5-positions of the phenyl ring of the acetophenone moiety of the compound of formula (IIa).

A particularly preferred compound of formula (IIa) is compound 22

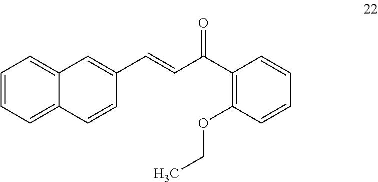 [SMILES: CCOc1ccccc1C(=O)/C=C/c1ccc2ccccc2c1]

or a pharmaceutically acceptable salt, solvate or prodrug thereof.

Furthermore, in formula (IIIa) shown above, the groups R¹, R², R⁵, R⁶, R⁷, R⁸, R⁹ and the variables n and m have the following meanings and preferred meanings:

R¹ and R² are each independently selected from hydrogen, halogen (such as, e.g., —F, —Cl, —Br, or —I), —NO₂, —CF₃, —ON, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). R¹ is preferably selected from hydrogen, halogen, —NO₂, —CF₃, —CN, C₁₋₄ alkyl, —OH, —O(C₁₋₄ alkyl), —SH, —S(C₁—2, alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl), more preferably from hydrogen, halogen, —NO₂, —OH, —O(C₁₋₄ alkyl), —SH, or —S(C₁₋₄ alkyl), and even more preferably R¹ is hydrogen. R² is preferably selected from hydrogen, halogen, —NO₂, —OH, —O(C₁₋₄ alkyl), —SH, or —S(C₁₋₄ alkyl), and more preferably R² is hydrogen. Accordingly, it is particularly preferred that R¹ and R² are each hydrogen.

Alternatively, R¹ and R² are mutually linked to form a group —C(R⁹)═C(R⁹)—, preferably a group —CH═CH—.

R⁵ and R⁶ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, R⁵ and R⁶ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). More preferably, R⁵ and R⁶ are each independently selected from hydrogen, halogen, —NO₂, —OH, —O(C₁₋₄ alkyl), —SH, or —S(C₁₋₄ alkyl). Even more preferably, R⁵ and R⁶ are each hydrogen.

Each R⁷ is independently selected from halogen, —NO₂, —CF₃, —CN, C₁₋₄ alkyl, —OH, —O(C alkyl), —SH, —S(C₁₋₄ alkyl), —NH₂, —NH(C₁₋₄ alkyl), —N(C₁₋₄ alkyl)(C₁₋₄ alkyl), —CO—NH₁₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, each R⁷ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl), —SO₂—NH₂, —SO₂—NH(C₁₋₄ alkyl), or —SO₂—N(C₁₋₄ alkyl)(C₁₋₄ alkyl). More preferably, each R⁷ is independently selected from halogen, —NO₂, —OH, —O(C₁₋₄ alkyl), —SH, or —S(C₁₋₄ alkyl). Even more preferably, each R⁷ is independently selected from halogen, —OH, —OCH₃, —OCH₂CH₃, —SH, —SCH₃, —SCH₂CH₃. If n is equal to or greater than 2, two groups R⁷ which are attached to adjacent carbon atoms may also be mutually linked to form a group —C(R⁹)═C(R⁹)—C(R⁹)═C(R⁹)— (such as, e.g., a group —CH═CH—CH═CH—) or a group —O—CH(R⁹)—O— (such as, e.g., a group —O—CH₂—O—).

Each R⁸ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, each R⁸ is independently selected from halogen, —NO₂, —CF₃, —ON, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). More preferably, each R⁸ is independently selected from halogen, —NO₂, —OH, —O(C₁₋₄ alkyl), —SH, or —S(C₁₋₄ alkyl). Even more preferably, each R⁸ is independently selected from halogen, —OH, —OCH₃, —OCH₂CH₃, —SH, —SCH₃, —SCH₂CH₃. If m is equal to or greater than 2, two groups R⁸ which are attached to adjacent carbon atoms may also be mutually linked to form a group —C(R⁹)═C(R⁹)—C(R⁹)═C(R⁹)— (such as, e.g., a group —CH═CH—CH═CH—) or a group —O—CH(R⁹)—O— (such as, e.g., a group —O—CH₂—O—).

Each R⁹ is independently selected from hydrogen, halogen, —NO₂, —CF₃, —ON, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(O₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁—4 alkyl). Preferably, each R⁹ is independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). More preferably, each R⁹ is independently selected from hydrogen, halogen, —NO₂, —OH, —O(C₁₋₄ alkyl), —SH, or —S(C₁₋₄ alkyl). Even more preferably, each R⁹ is independently selected from hydrogen, halogen, —OH, —OCH₃, —OCH₂CH₃, —SH, —SCH₃, —SCH₂CH₃. Most preferably, each R⁹ is hydrogen.

n is 0, 1, 2, 3, or 4. Preferably, n is 0, 1, 2, 3. More preferably, n is 0 or 1. Even more preferably, n is 1.

m is 0, 1, 2, 3, or 4. Preferably, m is 0, 1, 2, 3. More preferably, m is 1 or 3. In a particularly preferred embodiment, m is 1. In a further particularly preferred embodiment, m is 3.

Moreover, in formula (IVa) as defined above, the groups R¹, R², R⁵, R⁶, R⁷, R⁸, R⁹ and the variables n and m accordingly have the following meanings and preferred meanings:

R¹ and R² are each independently selected from hydrogen, halogen (such as, e.g., —F, —Cl, —Br, or —I), —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —B(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). R¹ is preferably selected from hydrogen, halogen, —NO₂, —CF₃, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl), more preferably from hydrogen, halogen, —NO₂, —OH, —O(C₁₋₄ alkyl), —SH, or —S(C₁₋₄ alkyl), and even more preferably R¹ is hydrogen. R² is preferably selected from hydrogen, halogen, —NO₂, —OH, —O(C₁₋₄ alkyl), —SH, or —S(C₁₋₄ alkyl), and more preferably R² is hydrogen. Accordingly, it is particularly preferred that R¹ and R² are each hydrogen.

Alternatively, R¹ and R² are mutually linked to form a group —C(R⁹)═C(R⁹)—, preferably a group —CH═CH—.

R⁵ and R⁶ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, R⁵ and R⁶ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). More preferably, R⁵ and R⁶ are each independently selected from hydrogen, halogen, —NO₂, —OH, —O(C₁₋₄ alkyl), —SH, or —S(C₁₋₄ alkyl). Even more preferably, R⁵ and R⁶ are each hydrogen.

Each R⁷ is independently selected from halogen, —NO₂, —CF₃, —CN, C₁₋₄ alkyl, —OH, —O(C₁₋₄ alkyl), —SH, —S(C₁—4 alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —ON, O₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, each R⁷ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). More preferably, each R⁷ is independently selected from halogen, —NO₂, —OH, —O(C₁₋₄ alkyl), —SH, or —S(C₁₋₄ alkyl). Even more preferably, each R⁷ is independently selected from halogen, —OH, —OCH₃, —OCH₂CH₃, —SH, —SCH₃, —SCH₂CH₃. If n is equal to or greater than 2, two groups R⁷ which are attached to adjacent carbon atoms may also be mutually linked to form a group —C(R⁹)═C(R⁹)—C(R⁹)═C(R⁹)— (such as, e.g., a group —CH═CH—CH═CH—) or a group —O—CH(R⁹)—O— (such as, e.g., a group —O—CH₂—O—).

Each R⁸ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, C₁₋₄ alkyl, —OH, —O(C₁, alkyl), —SH, —S(C₁₋₄. alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(O₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, each R⁸ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). More preferably, each R⁸ is independently selected from halogen, —NO₂, —OH, —O(C₁₋₄ alkyl), —SH, or —S(C₁₋₄ alkyl). Even more preferably, each R⁸ is independently selected from halogen, —OH, —OCH₃, —OCH₂CH₃, —SH, —SCH₃, —SCH₂CH₃. If m is equal to or greater than 2, two groups R⁸ which are attached to adjacent carbon atoms may also be mutually linked to form a group —C(R⁹)═C(R⁹)—C(R⁹)═C(R⁹)— (such as, e.g., a group —CH═CH—CH═CH—) or a group —O—CH(R⁹)—O— (such as, e.g., a group —O—CH₂—O—).

Each R⁹ is independently selected from hydrogen, halogen, —NO₂, —CF₃, —ON, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more (such as, e.g., one, two, three, or four), preferably one or two, more preferably one, groups independently selected from halogen, —NO₂, —CF₃, —CN, O₁₋₄ alkyl, —OH, —O(C₁₋₄ alkyl), —SH, alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). Preferably, each R⁹ is independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl). More preferably, each R⁹ is independently selected from hydrogen, halogen, —NO₂, —OH, —O(C₁₋₄ alkyl), —SH, or —S(C₁. alkyl). Even more preferably, each R⁹ is independently selected from hydrogen, halogen, —OH, —OCH₃, —OCH₂CH₃, —SH, —SCH₃, —SCH₂CH₃. Most preferably, each R⁹ is hydrogen.

n is 0, 1, 2, 3, or 4. Preferably, n is 0, 1, 2, 3. More preferably, n is 0 or 1. Even more preferably, n is 1.

m is 0, 1, 2, 3, or 4. Preferably, m is 0, 1, 2, 3. More preferably, m is 1 or 3. In a particularly preferred embodiment, m is 1. In a further particularly preferred embodiment, m is 3. As used herein, the term “alkyl” refers to a monovalent saturated aliphatic (i.e., non-aromatic) acyclic hydrocarbon group (i.e., a group consisting of carbon atoms and hydrogen atoms) which may be linear or branched and does not comprise any carbon-to-carbon double bond or any carbon-to-carbon triple bond. A “C₁₋₄ alkyl” denotes an alkyl group having 1 to 4 carbon atoms.

As used herein, the term “halogen” refers to —F, —Cl, —Br, or —I, and in particular to —F, —Cl, or —Br.

For a person skilled in the field of synthetic chemistry, various ways for the preparation of the compounds of the present invention, in particular the compounds of formula (I), (II), (III) or (IV), as well as the compounds of formula (Ia), (IIa), (IIIa) or (IVa), will be readily apparent.

For example, the compounds of the invention can be prepared in accordance with or in analogy to the synthetic routes described in the examples.

The scope of the invention embraces all pharmaceutically acceptable salt forms of the compounds of formula (I), (II), (III) or (IV), as well as the compounds of formula (Ia), (IIa), (IIIa) or (IVa), which may be formed, e.g., by protonation of an atom carrying an electron lone pair which is susceptible to protonation, such as an amino group, with an inorganic or organic acid, or as a salt of a carboxylic acid group with a physiologically acceptable cation as they are well-known in the art. Exemplary base addition salts comprise, for example, alkali metal salts such as sodium or potassium salts; alkaline earth metal salts such as calcium or magnesium salts; ammonium salts; aliphatic amine salts such as trimethylamine, triethylamine, dicyclohexylamine, ethanolamine, diethanolamine, triethanolamine, procaine salts, meglumine salts, diethanol amine salts or ethylenediamine salts; aralkyl amine salts such as N,N-dibenzylethylenediamine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts or lysine salts. Exemplary acid addition salts comprise, for example, mineral acid salts such as hydrochloride, hydrobromide, hydroiodide, sulfate salts, nitrate salts, phosphate salts (such as, e.g., phosphate, hydrogenphosphate, or dihydrogenphosphate salts), carbonate salts, hydrogencarbonate salts or perchlorate salts; organic acid salts such as acetate, propionate, butyrate, pentanoate, hexanoate, heptanoate, octanoate, cyclopentanepropionate, undecanoate, lactate, maleate, oxalate, fumarate, tartrate, malate, citrate, nicotinate, benzoate, salicylate or ascorbate salts; sulfonate salts such as methanesulfonate, ethanesulfonate, 2-hydroxyethanesulfonate, benzenesulfonate, p-toluenesulfonate (tosylate), 2-naphthalenesulfonate, 3-phenylsulfonate, or camphorsulfonate salts; and acidic amino acid salts such as aspartate or glutamate salts.

Moreover, the scope of the invention embraces solid forms of the compounds of formula (I), (II), (III) or (IV), as well as the compounds of formula (Ia), (IIa), (IIIa) or (IVa), in any solvated form, including e.g. solvates with water, for example hydrates, or with organic solvents such as, e.g., methanol, ethanol or acetonitrile, i.e. as a methanolate, ethanolate or acetonitrilate, respectively; or in the form of any polymorph.

Furthermore, the formulas in the present application are intended to cover all possible stereoisomers, including enantiomers and diastereomers, of the indicated compounds.

Thus, all stereoisomers of the compounds of the present invention, in particular the compounds of formula (I), (II), (III) or (IV), as well as the compounds of formula (Ia), (IIa), (IIIa) or (IVa), are contemplated as part of the present invention, either in admixture or in pure or substantially pure form. The scope of the compounds according to the invention embraces all the possible stereoisomers and their mixtures. It very particularly embraces the racemic forms and the isolated optical isomers. The racemic forms can be resolved by physical methods, such as, e.g., fractional crystallization, separation or crystallization of diastereomeric derivatives or separation by chiral column chromatography. The individual optical isomers can be obtained from the racemates using conventional methods, such as, e.g., salt formation with an optically active acid followed by crystallization.

Pharmaceutically acceptable prodrugs of compounds of the present invention, in particular of the compounds of formula (I), (II), (III) or (IV), as well as the compounds of formula (Ia), (IIa), (IIIa) or (IVa), are derivatives which have chemically or metabolically cleavable groups and become, by solvolysis or under physiological conditions, the compounds of the present invention which are pharmaceutically active in vivo. Prodrugs of compounds of the present invention may be formed in a conventional manner with a functional group of the compounds such as with an amino, hydroxy or carboxy group. The prodrug derivative form often offers advantages of solubility, tissue compatibility or delayed release in a mammalian organism (see, Bundgaard, H., Design of Prodrugs, pp. 7-9, 21-24, Elsevier, Amsterdam 1985). Prodrugs include acid derivatives well known to the person skilled in the art, such as, for example, esters prepared by reaction of the parent acidic compound with a suitable alcohol, or amides prepared by reaction of the parent acid compound with a suitable amine. When a compound of the present invention has a carboxyl group, an ester derivative prepared by reacting the carboxyl group with a suitable alcohol or an amide derivative prepared by reacting the carboxyl group with a suitable amine is exemplified as a prodrug. An especially preferred ester derivative as a prodrug is methylester, ethylester, n-propylester, isopropylester, n-butylester, isobutylester, tert-butylester, morpholinoethylester, N,N-diethylglycolamidoester or α-acetoxyethylester. When a compound of the present invention has a hydroxy group, an acyloxy derivative prepared by reacting the hydroxyl group with a suitable acylhalide or a suitable acid anhydride is exemplified as a prodrug. An especially preferred acyloxy derivative as a prodrug is —OC(═O)—CH₃, —OC(═O)—C₂H₅, —OC(═O)-(tert-Bu), —OC(═O)—C₁₅H₃₁, —OC(═O)-(m-COONa-Ph), —OC(═O)—CH₂CH₂COONa, —O(C═O)—CH(NH₂)CH₃ or —OC(═O)—CH₂—N(CH₃)₂. When a compound of the present invention has an amino group, an amide derivative prepared by reacting the amino group with a suitable acid halide or a suitable mixed anhydride is exemplified as a prodrug. An especially preferred amide derivative as a prodrug is —NHC(═O)—(CH₂)₂OCH₃ or —NHC(═O)—CH(NH₂)CH₃.

The compounds described herein may be administered as compounds per se in their use as pharmacophores or pharmaceutical compositions or may be formulated as medicaments. Within the scope of the present invention are pharmaceutical compositions comprising as an active ingredient a compound of formula (I), (II), (III) or (IV), as well as the compounds of formula (Ia), (IIa), (IIIa) or (IVa), as defined above. The pharmaceutical compositions may optionally comprise one or more pharmaceutically acceptable excipients, such as carriers, diluents, fillers, disintegrants, lubricating agents, binders, colorants, pigments, stabilizers, preservatives, or antioxidants.

The pharmaceutical compositions can be formulated by techniques known to the person skilled in the art, such as the techniques published in Remington's Pharmaceutical Sciences, 20^{th} Edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovule. Dosage forms for nasal administration can be administered via inhalation and insufflation, for example by a metered inhaler. Dosage forms for topical administration include creams, gels, ointments, salves, patches and transdermal delivery systems.

The compounds according to the invention, in particular the compounds of formula (I), (II), (III) or (IV), as well as the compounds of formula (Ia), (IIa), (IIIa) or (IVa), or the above described pharmaceutical compositions comprising one or more compounds of formula (I), (II), (III) or (IV), or pharmaceutical compositions comprising one or more compounds of formula (Ia), (IIa), (IIIa) or (IVa), may be administered to a subject by any convenient route of administration, whether systemically/peripherally or at the site of desired action, including but not limited to one or more of: oral (e.g. as a tablet, capsule, or as an ingestible solution), topical (e.g., transdermal, intranasal, ocular, buccal, and sublingual), parenteral (e.g., using injection techniques or infusion techniques, and including, for example, by injection, e.g. subcutaneous, intradermal, intramuscular, intravenous, intraarterial, intracardiac (i.e., intracardial), intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g. through mouth or nose), gastrointestinal, intrauterine, intraocular, subcutaneous, ophthalmic (including intravitreal or intracameral), rectal, and vaginal.

If said compounds or pharmaceutical compositions are administered parenterally, then examples of such administration include one or more of: intravenously, intraarterially, intraperitoneally, intrathecally, intraventricularly, intraurethrally, intrasternally, intracranially, intracardially, intramuscularly or subcutaneously administering the compounds pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Alternatively, said compounds or pharmaceutical compositions can be administered in the form of a suppository or pessary, or it may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds of formula (I), (II), (III) or (IV), as well as the compounds of formula (Ia), (IIa), (IIIa) or (IVa), for administration to a human (of approximately 70 kg body weight) may be 0.001 mg to 2000 mg, preferably 0.05 mg to 1000 mg, of the active ingredient per unit dose. The unit dose may be administered, for example, 1 to 4 times per day. The dose will depend on the route of administration. It will be appreciated that it may be necessary to make routine variations to the dosage depending on the age and weight of the patient/subject as well as the severity of the condition to be treated. The precise dose and route of administration will ultimately be at the discretion of the attendant physician or veterinarian.

The compounds of the present invention, in particular the compounds of formula (I), (II), (III) or (IV), as well as the compounds of formula (Ia), (IIa), (IIIa) or (IVa), can be used in combination with other therapeutic agents. When a compound of the invention is used in combination with a second therapeutic agent active against the same disease, the dose of each compound may differ from that when the compound is used alone. The combination of a compound of this invention with another drug may comprise the administration of the drug with the compound of the invention. Such an administration may comprise simultaneous/concomitant administration. However, also sequential/separate administration is envisaged, as discussed also below.

Preferably, the second therapeutic agent to be administered in combination with the compounds of this invention is an anticancer drug. The anticancer drug to be administered in combination with the compounds of this invention may be a tumor angiogenesis inhibitor (for example, a protease inhibitor, an epidermal growth factor receptor kinase inhibitor, or a vascular endothelial growth factor receptor kinase inhibitor); a cytotoxic drug (for example, an antimetabolite, such as purine and pyrimidine analogue antimetabolites); an antimitotic agent (for example, a microtubule stabilizing drug or an antimitotic alkaloid); a platinum coordination complex; an anti-tumor antibiotic; an alkylating agent (for example, a nitrogen mustard or a nitrosourea); an endocrine agent (for example, an adrenocorticosteroid, an androgen, an anti-androgen, an estrogen, an anti-estrogen, an aromatase inhibitor, a gonadotropin-releasing hormone agonist, or a somatostatin analogue); or a compound that targets an enzyme or receptor that is overexpressed and/or otherwise involved in a specific metabolic pathway that is misregulated in the tumor cell (for example, ATP and GTP phosphodiesterase inhibitors, histone deacetylase inhibitors, protein kinase inhibitors (such as serine, threonine and tyrosine kinase inhibitors (for example, Abelson protein tyrosine kinase)) and the various growth factors, their receptors and kinase inhibitors therefor (such as epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors)); methionine, aminopeptidase inhibitors, proteasome inhibitors, cyclooxygenase inhibitors (for example, cyclooxygenase-1 or cyclooxygenase-2 inhibitors) and topoisomerase inhibitors (for example, topoisomerase I inhibitors or topoisomerase II inhibitors).

An alkylating agent which can be used as an anticancer drug in combination with a compound of the present invention may be, for example, a nitrogen mustard (such as cyclophosphamide, mechlorethamine (chlormethine), uramustine, melphalan, chlorambucil, ifosfamide, bendamustine, or trofosfamide), a nitrosourea (such as carmustine, streptozocin, fotemustine, lomustine, nimustine, prednimustine, ranimustine, or semustine), an alkyl sulfonate (such as busulfan, mannosulfan, or treosulfan), an azindine (such as hexamethylmelamine (altretamine), triethylenemelamine, ThioTEPA (N,N′N′-triethylenethiophosphoramide), carboquone, or triaziquone), a hydrazine (such as procarbazine), a triazene (such as dacarbazine), or an imidazotetrazines (such as temozolomide).

A platinum coordination complex which can be used as an anticancer drug in combination with a compound of the present invention may be, for example, cisplatin, carboplatin, nedaplatin, oxaliplatin, atraplatin, or triplatin tetranitrate.

A cytotoxic drug which can be used as an anticancer drug in combination with a compound of the present invention may be, for example, an antimetabolite, including folic acid analogue antimetabolites (such as aminopterin, methotrexate, pemetrexed, or raltitrexed), purine analogue antimetabolites (such as cladribine, clofarabine, fludarabine, 6-mercaptopurine (including its prodrug form azathioprine), pentostatin, or 6-thioguanine), and pyrimidine analogue antimetabolites (such as cytarabine, decitabine, 5-fluorouracil (including its prodrug forms capecitabine and tegafur), floxuridine, gemcitabine, enocitabine, or sapacitabine).

An antimitotic agent which can be used as an anticancer drug in combination with a compound of the present invention may be, for example, a taxane (such as docetaxel, larotaxel, ortataxel, paclitaxel/taxol, or tesetaxel), a Vinca alkaloid (such as vinblastine, vincristine, vinflunine, vindesine, or vinorelbine), an epothilone (such as epothilone A, epothilone B, epothilone C, epothilone D, epothilone E, or epothilone F) or an epothilone B analogue (such as ixabepilone/azaepothilone B).

An anti-tumor antibiotic which can be used as an anticancer drug in combination with a compound of the present invention may be, for example, an anthracycline (such as aclarubicin, daunorubicin, doxorubicin, epirubicin, idarubicin, amrubicin, pirarubicin, valrubicin, or zorubicin), an anthracenedione (such as mitoxantrone, or pixantrone) or an anti-tumor antibiotic isolated from Streptomyces (such as actinomycin (including actinomycin D), bleomycin, mitomycin (including mitomycin C), or plicamycin).

A tyrosine kinase inhibitor which can be used as an anticancer drug in combination with a compound of the present invention may be, for example, axitinib, bosutinib, cediranib, dasatinib, erlotinib, gefitinib, imatinib, lapatinib, lestaurtinib, nilotinib, semaxanib, sorafenib, sunitinib, or vandetanib.

A topoisomerase-inhibitor which can be used as an anticancer drug in combination with a compound of the present invention may be, for example, a topoisomerase I inhibitor (such as irinotecan, topotecan, camptothecin, belotecan, rubitecan, or lamellarin D) or a topoisomerase II inhibitor (such as amsacrine, etoposide, etoposide phosphate, teniposide, or doxorubicin).

Further anticancer drugs may be used in combination with a compound of the present invention. The anticancer drugs may comprise biological or chemical molecules, like TNF-related apoptosis-inducing ligand (TRAIL), tamoxifen, amsacrine, bexarotene, estramustine, irofulven, trabectedin, cetuximab, panitumumab, tositumomab, alemtuzumab, bevacizumab, edrecolomab, gemtuzumab, alvocidib, seliciclib, aminolevulinic acid, methyl aminolevulinate, efaproxiral, porfimer sodium, talaporfin, temoporfin, verteporfin, alitretinoin, tretinoin, anagrelide, arsenic trioxide, atrasentan, bortezomib, carmofur, celecoxib, demecolcine, elesclomol, elsamitrucin, etoglucid, lonidamine, lucanthone, masoprocol, mitobronitol, mitoguazone, mitotane, oblimersen, omacetaxine, sitimagene, ceradenovec, tegafur, testolactone, tiazofurine, tipifarnib, and vorinostat.

Also biological drugs, like antibodies, antibody fragments, antibody constructs (for example, single-chain constructs), and/or modified antibodies (like CDR-grafted antibodies, humanized antibodies, “full humanized” antibodies, etc.) directed against cancer or tumor markers/factors/cytokines involved in proliferative diseases can be employed in co-therapy approaches with the compounds of the invention. Examples of such biological molecules are anti-HER2 antibodies (e.g. trastuzumab, Herceptin®), anti-CD20 antibodies (e.g. Rituximab, Rituxan®, MabThera®, Reditux®), anti-CD19/CD3 constructs (see, e.g., EP-B1 1071752) and anti-TNF antibodies (see, e.g., Taylor PC. Antibody therapy for rheumatoid arthritis. Curr Opin Pharmacol. 2003. 3(3):323-328). Further antibodies, antibody fragments, antibody constructs and/or modified antibodies to be used in co-therapy approaches with the compounds of the invention can be found in Taylor PC. Curr Opin Pharmacol. 2003. 3(3):323-328; Roxana A. Maedica. 2006. 1(1):63-65.

The combinations referred to above may conveniently be presented for use in the form of a pharmaceutical formulation. The individual components of such combinations may be administered either sequentially or simultaneously/concomitantly in separate or combined pharmaceutical formulations by any convenient route. When administration is sequential, either the present compound or the second therapeutic agent may be administered first. When administration is simultaneous, the combination may be administered either in the same or different pharmaceutical composition. When combined in the same formulation it will be appreciated that the two compounds must be stable and compatible with each other and the other components of the formulation. When formulated separately they may be provided in any convenient formulation, conveniently in such manner as are known for such compounds in the art.

The compounds of the present invention, in particular the compounds of formula (I), (II), (III) or (IV), as well as the compounds of formula (Ia), (IIa), (IIIa) or (IVa), can also be administered in combination with physical therapy, such as radiotherapy. Radiotherapy may commence before, after, or simultaneously with administration of the compounds. For example, radiotherapy may commence 1-10 minutes, 1-10 hours or 24-72 hours after administration of the compounds. Yet, these time frames are not to be construed as limiting. The subject is exposed to radiation, preferably gamma radiation, whereby the radiation may be provided in a single dose or in multiple doses that are administered over several hours, days and/or weeks. Gamma radiation may be delivered according to standard radiotherapeutic protocols using standard dosages and regimens. Again, and without being bound by theory, the compounds of the present invention may be used to render cells, in particular undesired proliferative/hyperproliferative cells like cancer or tumor cells, more susceptible to such a physical therapy, e.g. radiotherapy.

Accordingly, the present invention relates to a compound of formula (I), (II), (III) or (IV), as well as the compounds of formula (Ia), (IIa), (IIIa) or (IVa), or a pharmaceutically acceptable salt, solvate, or prodrug thereof, or a pharmaceutical composition comprising any of the aforementioned entities in combination with a pharmaceutically acceptable excipient, for use in the treatment or prevention of cancer, in particular the treatment or prevention of a malignant hematological disease/disorder, whereby the compound or the pharmaceutical composition is to be administered in combination with an anti-proliferative drug, an anticancer drug, a cytostatic drug, a cytotoxic drug and/or radiotherapy.

The term “treatment of a disorder or disease” as used herein, such as “treatment of cancer”, is well known in the art. “Treatment of a disorder or disease” implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

“Treatment of a disorder or disease” may, for example, lead to a halt in the progression of the disorder or disease (e.g., no deterioration of symptoms) or a delay in the progression of the disorder or disease (in case the halt in progression is of a transient nature only). “Treatment of a disorder or disease” may also lead to a partial response (e.g., amelioration of symptoms) or complete response (e.g., disappearance of symptoms) of the subject/patient suffering from the disorder or disease. “Amelioration” of a disorder or disease may, for example, lead to a halt in the progression of the disorder or disease or a delay in the progression of the disorder or disease. Such a partial or complete response may be followed by a relapse. It is to be understood that a Subject/patient may experience a broad range of responses to a treatment (e.g., the exemplary responses as described herein above).

Treatment of a disorder or disease may, inter alia, comprise curative treatment (preferably leading to a complete response and eventually to healing of the disorder or disease) and palliative treatment (including symptomatic relief).

Also the term “prevention of a disorder or disease” as used herein, such “prevention of cancer”, is well known in the art. For example, a patient/subject suspected of being prone to suffer from a disorder or disease as defined herein may, in particular, benefit from a prevention of the disorder or disease. The subject/patient may have a susceptibility or predisposition for a disorder or disease, including but not limited to hereditary predisposition. Such a predisposition can be determined by standard assays, using, for example, genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of compounds of the present invention before any clinical and/or pathological symptoms are diagnosed or determined or can be diagnosed or determined by the attending physician.

The subject or patient, such as the subject in need of treatment, prevention or amelioration, may be a eukaryote, an animal, a vertebrate animal, a mammal, a rodent (e.g. a guinea pig, a hamster, a rat, a mouse), a murine (e.g. a mouse), a canine (e.g. a dog), a feline (e.g. a cat), an equine (e.g. a horse), a primate, a simian (e.g. a monkey or ape), a monkey (e.g. a marmoset, a baboon), an ape (e.g. gorilla, chimpanzee, orangutang, gibbon), or a human. The meaning of the terms “eukaryote”, “animal”, “mammal”, etc. is well known in the art and can, for example, be deduced from Wehner und Gehring (1995; Thieme Verlag). In the context of this invention, it is particularly envisaged that animals are to be treated which are economically, agronomically or scientifically important. Scientifically important organisms include, but are not limited to, mice, rats, and rabbits. Lower organisms such as, e.g., fruit flies like Drosophila melaaonaster and nematodes like Caenorhabditis elegans may also be used in scientific approaches. Non-limiting examples of agronomically important animals are sheep, cattle and pig, while, for example, cats and dogs may be considered as economically important animals. Preferably, the subject/patient is a mammal; more preferably, the subject/patient is a human.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention is also illustrated by the following illustrative figures. The appended figures show:

FIG. 1. Lead structure optimization of compound 1 resulted in the highly cytotoxic compound 22.

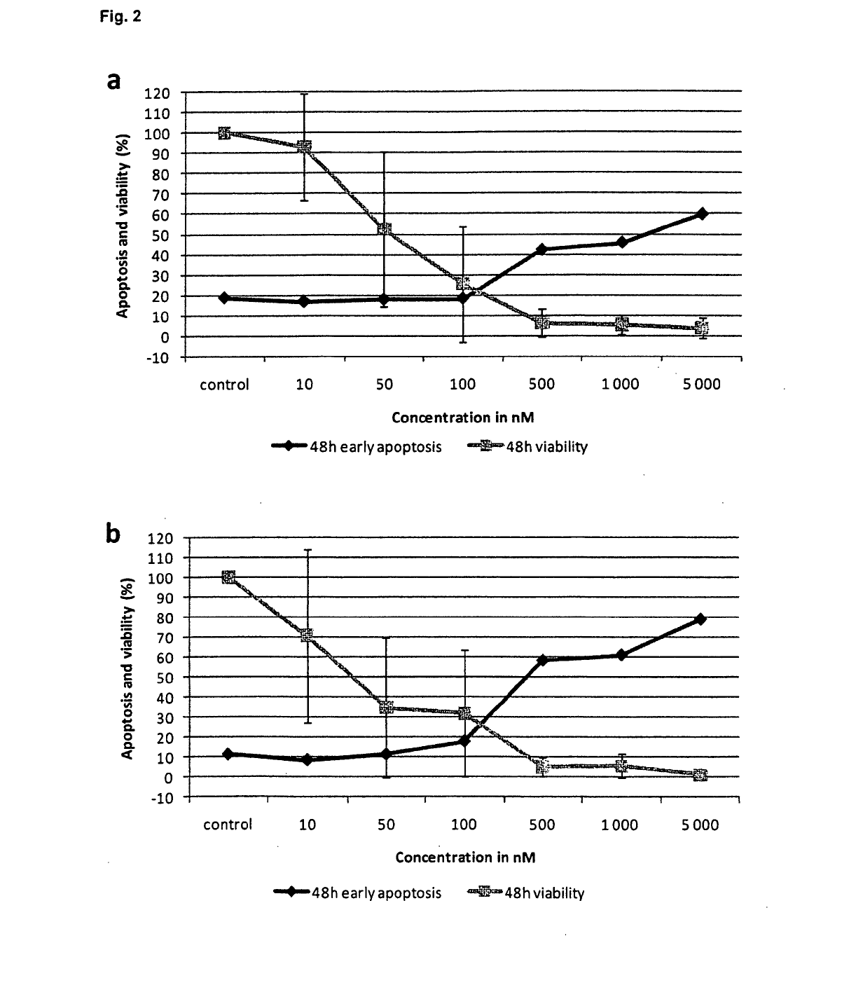

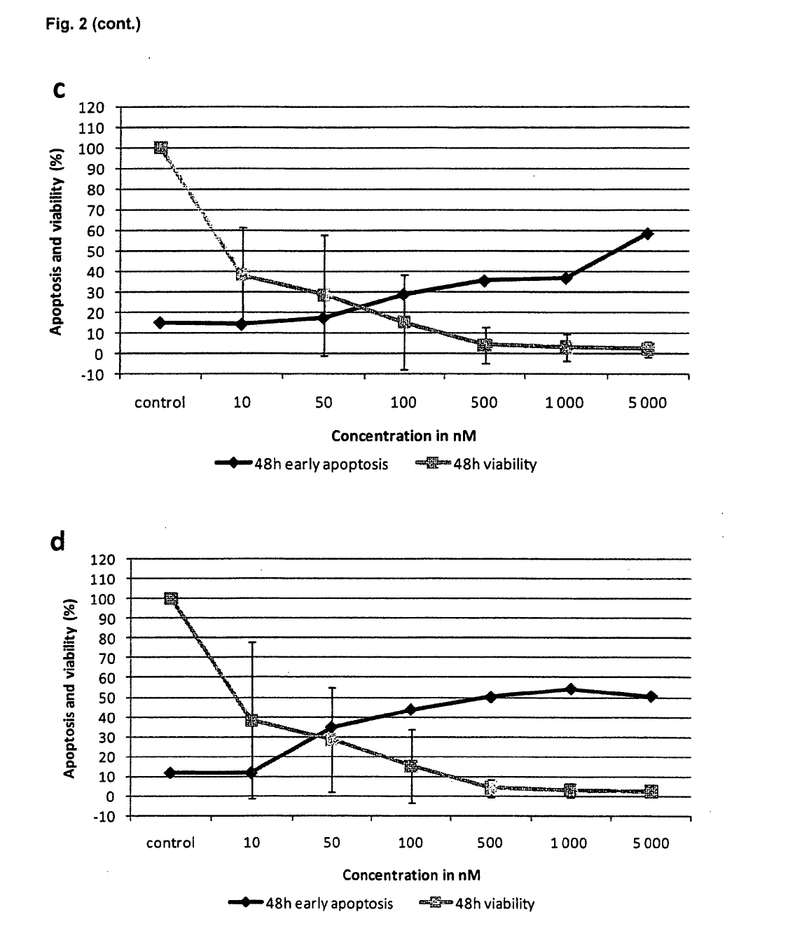

FIG. 2. Changes in early apoptosis in SU-DHL-6 or SU-DHL-9 NHL cells after 48 h incubation with compounds 1 and 22, respectively. Effect of compound 1 in a) SU-DHL-6 and b) SU-DHL-9, and effect of compound 22 in c) SU-DHL-6 and d) SU-DHL-9. Apoptosis was detected using propidium iodide/Annexin V FACS staining (Bender MedSystems). Viability was detected using the EZ4U viability kit (Biomedica).

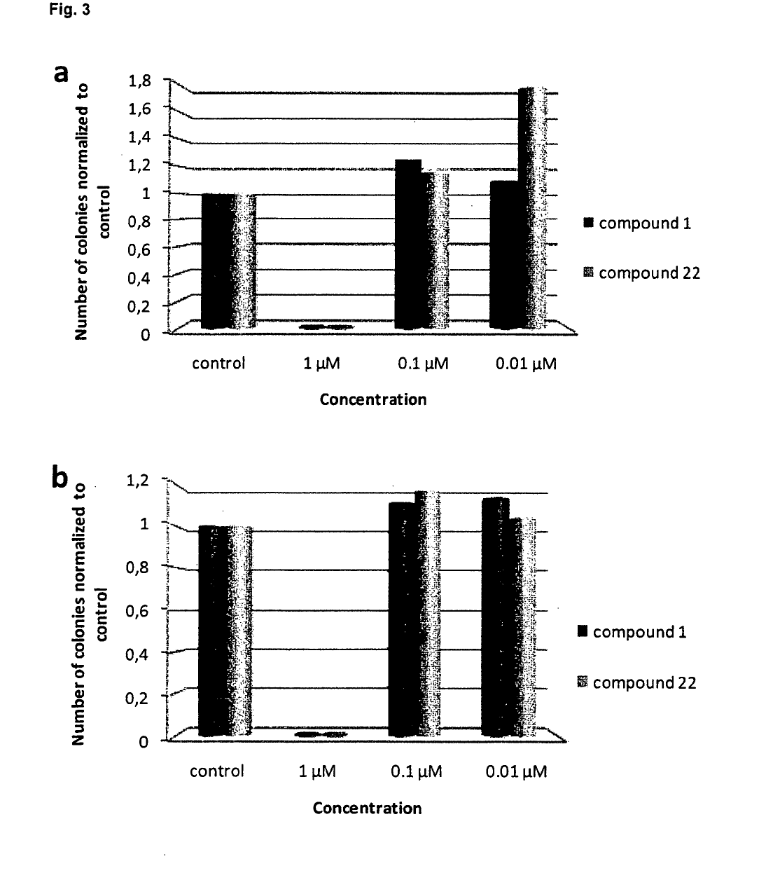

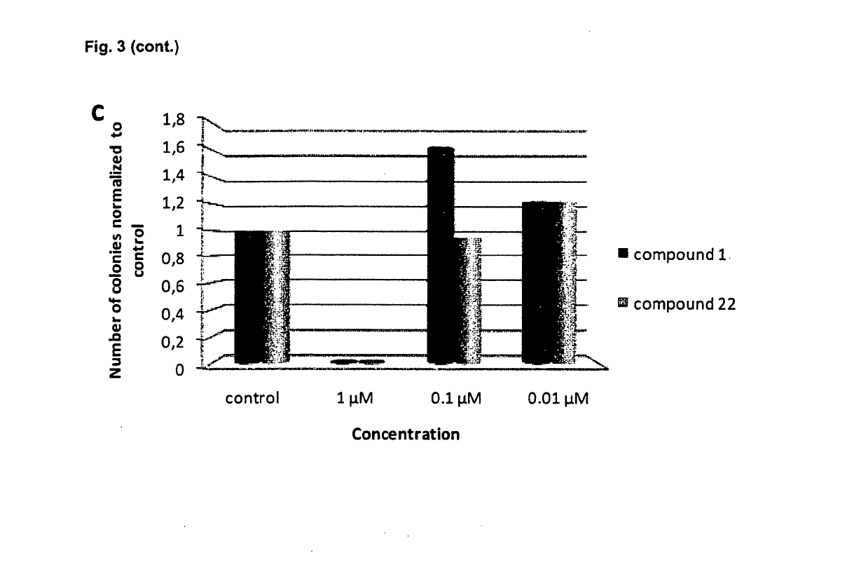

FIG. 3. Results of a standard colony forming assay in normal human hematopoietic progenitor cells. a: CFU-GM (myelomonocytic stern cells), b: BFU-E (erythroid stem cells), c: CFU-GEMM (myeloid stem cells) of compound 1 and 22, respectively. Cells were incubated with the compounds for 14 days.

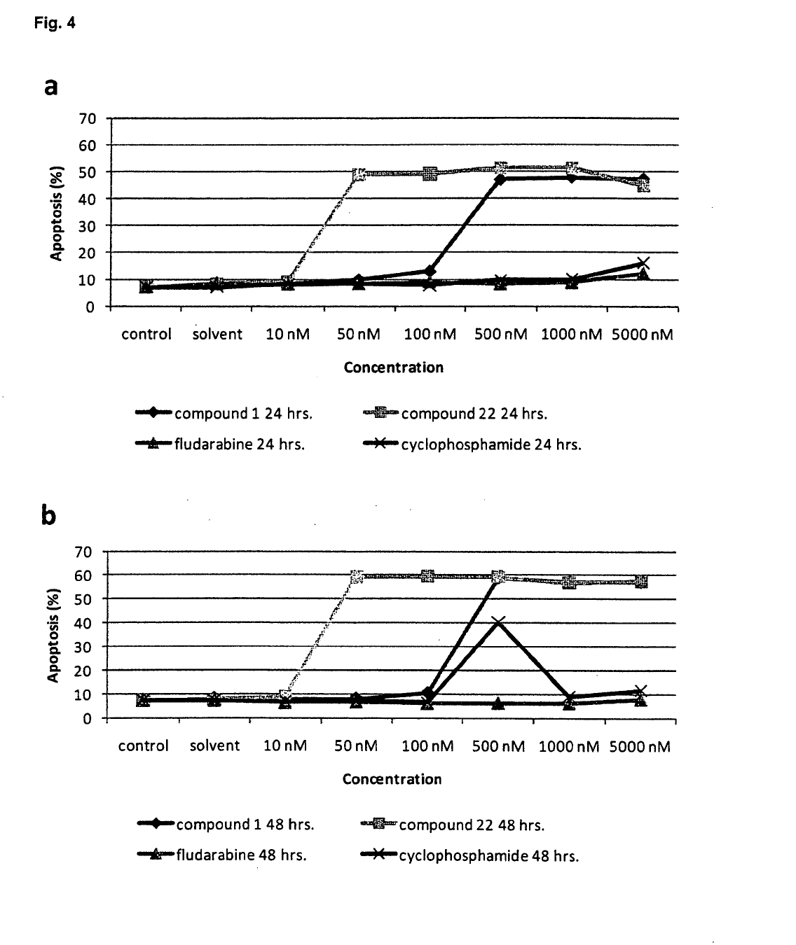

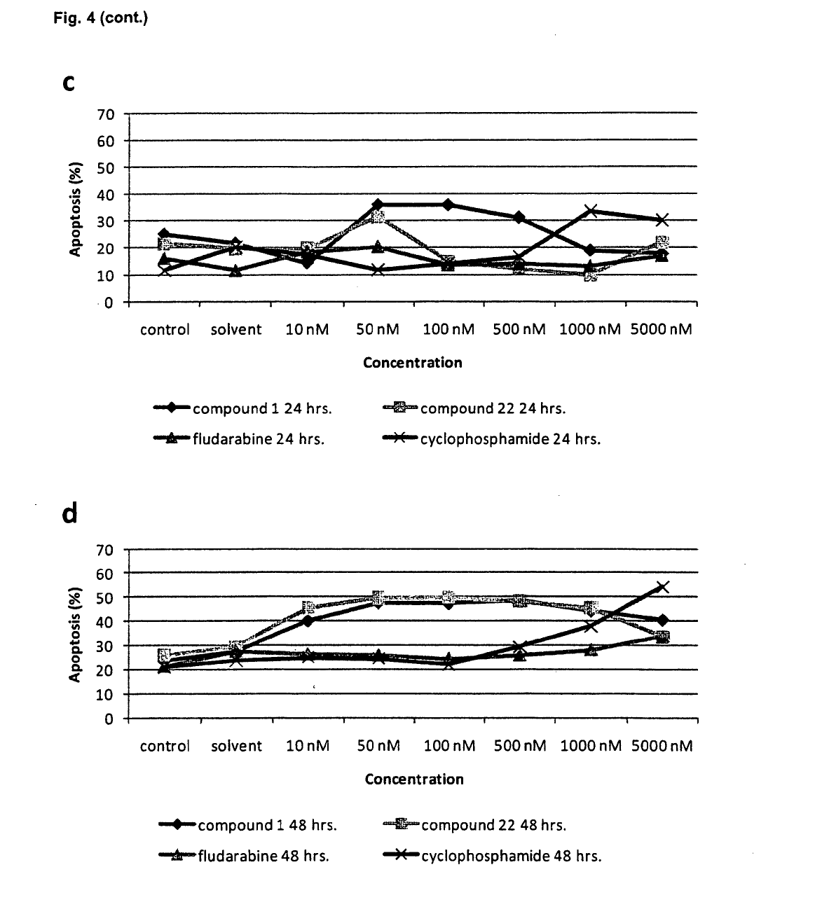

FIG. 4: Changes in early apoptosis in MEC-1 cells after 24 hours (a) and 48 hours (b) or in MEC-2 cells after 24 hours (c) and 48 hours (d) incubation with compounds 1 and 22, respectively. Apoptosis was detected using propidium iodide/Annexin V FACS staining (Bender MedSystems).

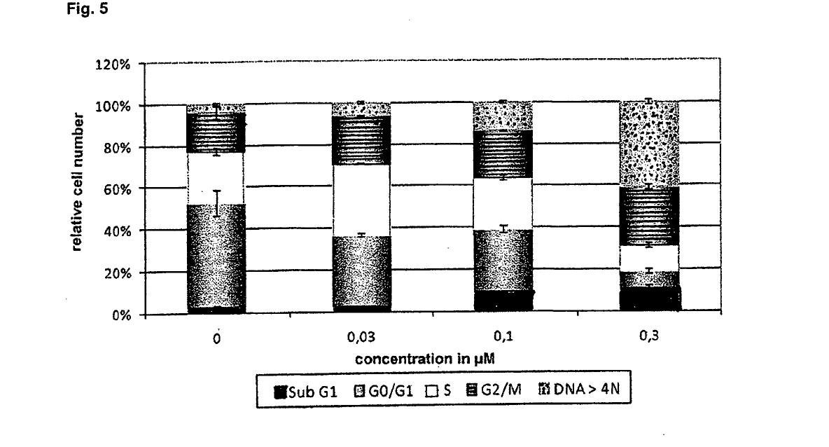

FIG. 5: Cell cycle analysis using SW480 cells treated with 0 μM (control using DMSO), 0.03 μM, 0.1 μM or 0.3 μM of compound 22.

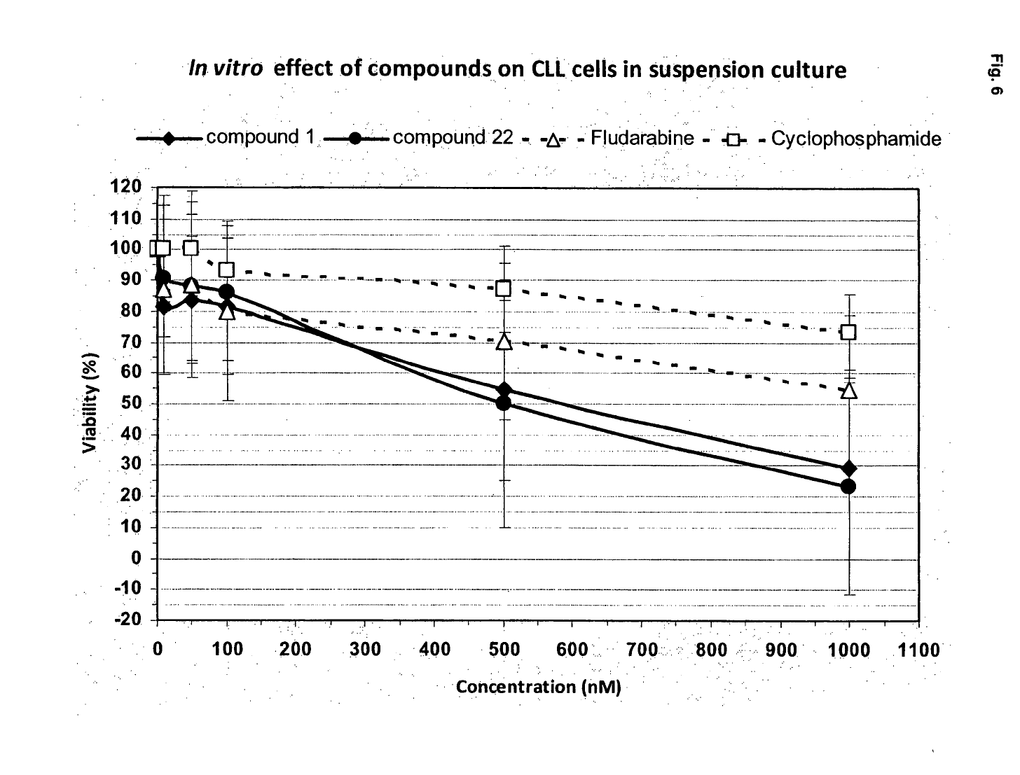

FIG. 6: Effect of compounds 1 and 22 according to the invention as well as the control drugs fludarabine and cyclophosphamide on the viability of chronic lymphocytic leukemia (CLL) cells in suspension culture after 48h incubation. Data points represent the arithmetic mean of 15 patients calculated in relation to the control. Viability was measured using the CellTiter Blue assay (Promega, Madison, Wis., USA), and cells incubated in solvent only were used as control.

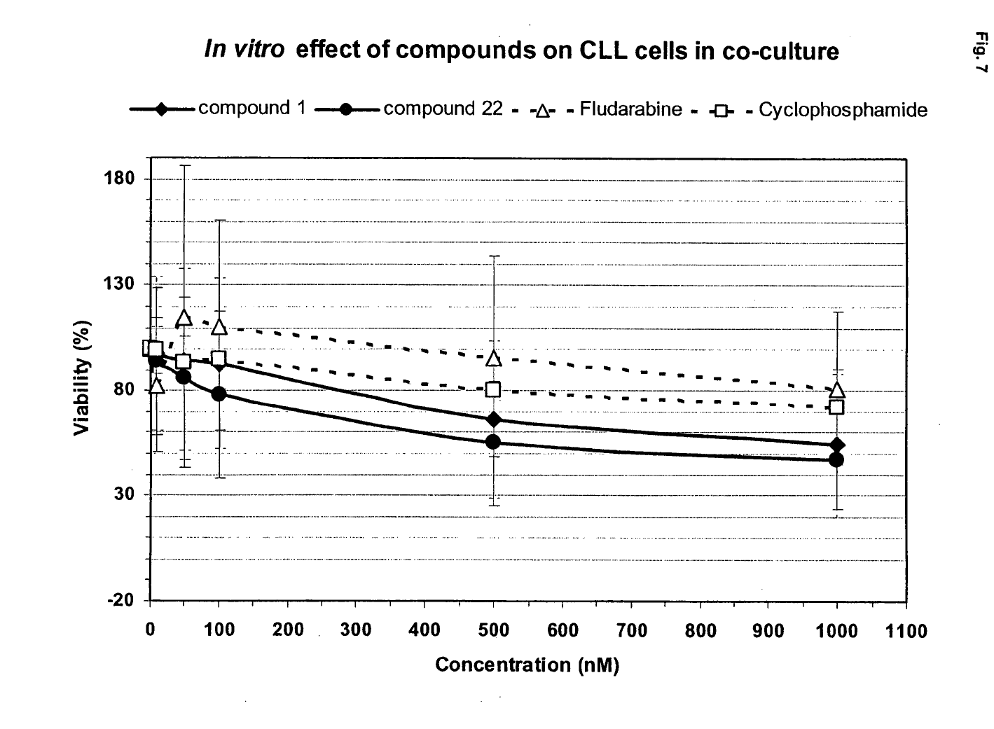

FIG. 7: Effect of compounds 1 and 22 according to the invention as well as the control drugs fludarabine and cyclophosphamide on the viability of chronic lymphocytic leukemia (CLL) cells in co-culture after 48 h incubation. Data points represent the arithmetic mean of 5 patients calculated in relation to the control. Viability was measured using the CellTiter Blue assay (Promega, Madison, Wis., USA), and cells incubated in solvent only were used as control.

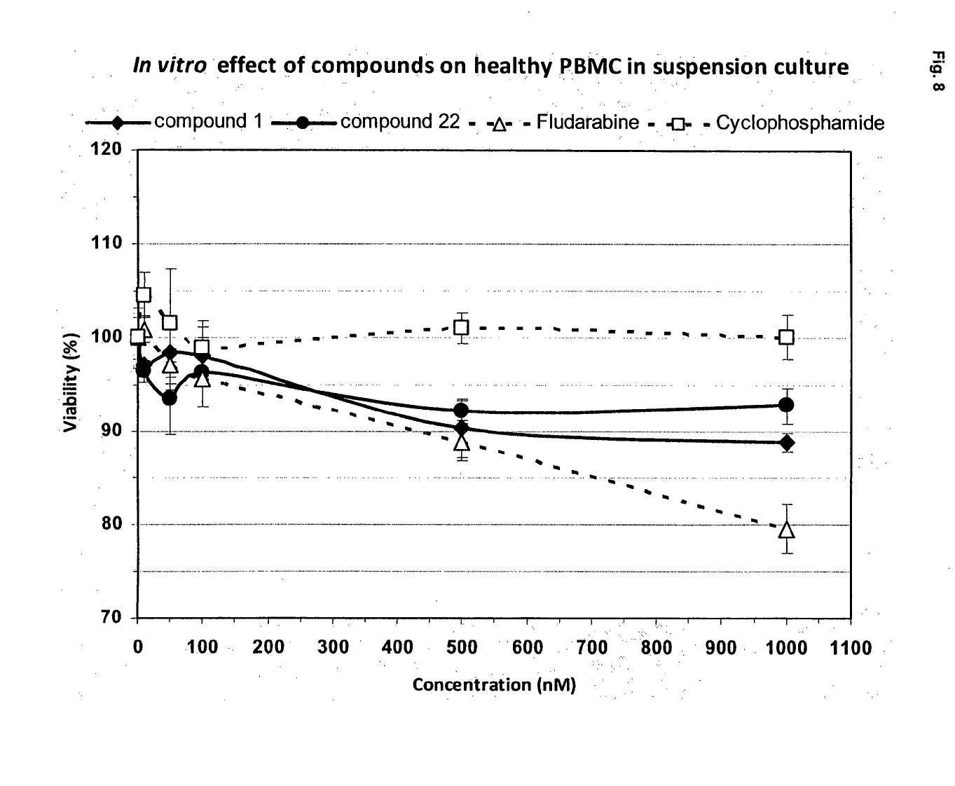

FIG. 8: Effect of compounds 1 and 22 according to the invention as well as the control drugs fludarabine and cyclophosphamide on the viability of healthy peripheral blood mononuclear cells (PBMC) in suspension culture after 48 h incubation. Data points represent one individual calculated in relation to the control. Viability was measured using the CellTiter Blue assay (Promega, Madison, Wis., USA), and cells incubated in solvent only were used as control.

The invention will now be described by reference to the following examples which are merely illustrative and are not to be construed as a limitation of the scope of the present invention.

### EXAMPLES

All chemicals obtained from commercial suppliers were used as received and were of analytical grade. Melting points were determined on a Kofler hot stage apparatus and are uncorrected. The ¹H- and ¹³C-NMR spectra were recorded on a Bruker Avance DPx200 (200 and 50 MHz). Chemical shifts are reported in δ units (ppm) relative to Me₄Si line as internal standard and J values are reported in Hertz. Mass spectra were obtained by a Hewlett Packard (MS: 5970) spectrometer. Solutions in organic solvents were dried over anhydrous sodium sulphate. The ¹H—, ¹³C-NMR and mass spectra of the new synthesized compounds are in agreement with the assigned structures. C, H, N analysis data were within ±0.4% of the theoretical values. Column chromatography was performed using silica gel 60, 70-230 mesh ASTM (Merck).

### Example 1

### Synthesis of Compounds According to the Invention and Intermediates

### Synthesis of Cinnamic Aldehydes

For the synthesis of cinnamic aldehydes two different methods (A and B) were used.

### Method A:

50.0 mmol of the corresponding cinnamic acid were dissolved in 200 ml methanol. The solution was rinsed with HCl gas for 15 min and then stirred at room temperature over night. The solvent was evaporated in vacuo and the ester was extracted with ethyl acetate. The organic layer was washed with 5% K₂CO₃ and brine, dried over sodium sulfate and evaporated in vacuo.

13 mmol of the so-obtained methyl ester were dissolved in 80 ml THF (dried over sodium) under argon atmosphere. The solution was cooled down to −78° C. 40 mmol of an 1 M DIBAL-H solution in toluene were injected during 40 min. The reaction solution was then stirred for 2 h at −78° C. Careful adding of 70 ml 10% NH₄Cl obtained a gelatinous substance, which was dissolved in 2N HCl. The product was extracted with ethyl acetate. The organic layer was washed with brine, dried over sulfate and evaporated in vacuo.

11.5 mmol of the so-obtained alcohol were dissolved in 20 ml toluene. 69 mmol MnO₂ were added, and the solution was stirred at room temperature for 30 h. The solution was filtered twice to remove an organic parts, and the solvent was evaporated in vacuo. The desired product was purified by using column chromatography.

### Method B:

5 mmol of the corresponding benzaldehyde derivative, 7.5 mmol (1,3-dioxolan-2-yl-methyl)triphenylphosphonium bromide and 5 mg 18-crown-6 were suspended in 15 ml anhydrous THF. 20.8 mmol sodium hydride was added carefully and the reaction mixture was stirred at room temperature till the reaction was completed (monitored by TLC). After cooling the mixture to 0° C., water and then 10% HCl were added carefully till the reaction mixture was slightly acidic. 60 minutes later, the solution was washed with 10% HCl and water, the organic solvent removed in vacuo and the crude product purified by flash chromatography.

### General Method for the Synthesis of Vinylogous Chalcone Derivatives

2.5 mmol of the appropriate acetophenone, indanone or tetralone derivative and 2 ml 50% sodium hydroxide were dissolved in ethanol and stirred for 30 minutes. Then, 2.5 mmol of the corresponding cinnamic aldehyde derivative, dissolved in 1 ml ethanol, were added and the reaction mixture was allowed to stir till the reaction was completed (monitored by TLC). The reaction mixture was poured into ice-water and acidified with 10% HCl. The so-obtained precipitate was filtered off and the crude product was recrystallized in ethanol.

### 5-(2-Methoxyphenyl)-1-(3′,4′,5′-trimethoxyphenyl)-2,4-pentadiene-1-one (1)

Compound 1 was synthesized from 2.5 mmol (0.525 g) 3,4,5-trimethoxyacetophenone and 2.5 mmol (0.410 g) 2-methoxycinnamaldehyde. Yield: 0.62 g (70.1%). Mp: 111° C. ¹H-NMR (CDCl₃, 200 MHz): δ 7.72-7.53 (m, 2H), 7.52-7.23 (m, 4H), 7.17-6.89 (m, 4H), 3.95 (s, 6H), 3.93 (s, 3H), 3.90 (s, 3H) ppm. ¹³C-NMR (CDCl₃, 50 MHz): δ 189.2, 157.6, 153.0 (2C), 145.9, 142.1, 137.3, 133.7, 130.4, 127.4 (2C), 125.0, 124.3, 120.7, 111.1, 105.8 (2C), 60.9, 56.3 (2C), 55.5 ppm. MS: m/z 354 (M⁺, 100%), 339 (73%), 195 (28%), 140 (25%), 121 (56%), 91 (54%). CHN analysis: calculated for C₂₁H₂₂O₅×0.3H₂O: C, 71.16%, H, 6.26%. found: C, 70.91%, H, 6.47%.

### (E)-2-Methylthiocinnamic aldehyde

This cinnamic aldehyde derivative was synthesized according to method B from 5 mmol (0.76 g; 0.64 ml) 2-methylthiobenzaldehyd. Yield: 0.66 g (90.4%). Mp: 74-76° C. ¹H-NMR (CDCl₃, 200 MHz): δ9.75 (AB system J_{AB}=7.8 Hz, 1H), 8.03 (AB system J_{AB}=15.8 Hz, 1H), 7.69-7.51 (m, 1H), 7.49-7.15 (m, 3H), 6.67 (two AB systems, J_{AB}=15.8 Hz, J_{AB}=7.8 Hz, 1H), 2.51 (s, 3H) ppm. ¹³C-NMR (CDCl₃, 50 MHz): δ 193.9, 149.2, 139.8, 133.0, 131.2, 129.9, 127.6, 127.3, 125.8, 16.7 ppm. MS: m/z 178 (M⁺, 9%), 149 (75%), 134 (100%), 131 (95%), 116 (12%), 91 (33%), 77 (19%). CHN analysis: calculated for C₁₀H₁₀OS: C, 67.38%, H, 5.66%. found: C, 67.60%, H, 5.45%.

### 5-(2-Methylthiophenyl)-1-(3′,4′,5′-trimethoxyphenyl)-2,4-pentadiene-1-one (3)

Compound 3 was synthesized from 2.5 mmol (0.525 g) 3,4,5-trimethoxyacetophenone and 2.5 mmol (0.456 g) (E)-2-methylthiocinnamaldehyde. Yield: 0.51 g (54.7%). Mp: 114.5-115.5° C. ¹H-NMR (CDCl₃, 200 MHz): δ 7.76-7.42 (m, 3H), 7.40-6.86 (m, 7H), 3.95 (s, 6H), 3.94 (s, 3H), 2.48 (s, 3H) ppm. ¹³C-NMR (CDCl₃, 50 MHz): 8189.0, 153.1 (2C), 144.8, 142.3, 138.6, 138.3, 135.1, 133.4, 129.4, 128.4, 127.3, 126.2, 125.6, 125.2, 105.8 (2C), 60.9, 56.3 (2C), 16.5 ppm. MS: m/z 370 (M⁺, 65%), 355 (46%), 297 (60%), 187 (100%), 149 (90%), 134 (84%), 115 (51%). CHN analysis: calculated for C₂₁H₂₂O₄S: C, 68.08%, H, 5.99%. found: C, 67.74%, H, 6.01%.

### 3-(2-Naphthyl)-1-(2′-ethoxyphenyl)-2-propene-1-one (22)

Compound 22 was synthesized from 2.5 mmol (0.411 g) 2-ethoxyacetophenone and 2.5 mmol (0.391 g) 2-naphthaldehyde. Yield: 0.21 g (27.1%). Mp: 96-97° C. ¹H-NMR (CDCl₃, 200 MHz): δ8.14-7.39 (m, 11H), 7.21-6.85 (m, 2H), 4.14 (q, J=6.9 Hz, 2H), 1.43 (t, J=6.9 Hz, 3H) ppm. ¹³C-NMR (CDCl₃, 50 MHz): 8192.7, 157.7, 142.6, 134.2, 133.4, 133.0, 132.8, 130.6, 130.3, 129.3, 128.6, 128.5, 127.7, 127.4, 127.1, 126.6, 123.6, 120.7, 112.6, 64.2, 14.8 ppm. MS: m/z 302 (M⁺, 13%), 287 (2%), 273 (3%), 257 (5%), 229 (7%), 151 (77%), 68 (100%). CHN analysis: calculated for C₂₁H₁₈O₂×0.2H₂O: C, 82.44%, H, 6.06%. found: C, 82.07%, H, 5.62%.

### 2-[3-(4-Dimethylaminophenyl)-prop-2-en-ylidene]-5,6-dimethoxyindan-1-one (28)

Compound 28 was synthesized from 2.5 mmol (0.481 g) 5,6-dimethoxy-1-indanone and 2.5 mmol (0.438 g) 4-(N,N)-dimethylaminocinnamaldehyde. Yield: 0.42 g (48.3%). Mp: 210-213° C. ¹H-NMR (CDCl₃, 200 MHz): δ 7.50-7.29 (m, 4H), 7.02-6.61 (m, 5H), 3.99 (s, 3H), 3.93 (s, 3H), 3.72 (s, 2H), 3.01 (s, 6H) ppm. ¹³C-NMR (CDCl₃, 50 MHz): 8192.5, 154.8, 150.9, 149.4, 143.8, 142.2, 134.1, 133.3, 132.7, 128.7 (20), 124.6, 119.9, 112.0 (2C), 107.2, 104.8, 56.2, 56.1, 40.2 (2C), 30.2 ppm. MS: m/z 349 (M⁺, 100%), 334 (17%), 290 (7%), 175 (19%), 144 (25%), 129 (20%), 117 (27%). CHN analysis: calculated for C₂₂H₂₃NO₃: C, 75.62%, H, 6.64%, N, 4.01%. found: C, 75.35%, H, 6.60%, N, 3.96%.

### 6-Methoxy-2-[(E,2E|E,2Z)-3-(3,4,5-trimethoxyphenyl)-2-propenylidene]-1-indanone (34)

Compound 34 was synthesized from 2.5 mmol (0.404 g) 6-methoxy-1-indanone and 2.5 (0.557 g) 3,4,5-trimethoxycinnamaldehyde. Yield: 0.44 g (48.6%). Mp: 182-186° C. ¹H-NMR (CDCl₃, 200 MHz): δ 7.51-7.29 (m, 3H), 7.19 (dd, ³J=8.3 Hz, ⁴J=2.5 Hz, 1H), 7.02-6.82 (m, 2H), 6.74 (s, 2H), 3.93 (s, 6H), 3.89 (s, 3H), 3.86 (s, 3H), 3.84-3.78 (m, 2H) ppm. ¹³C-NMR (CDCl₃, 50 MHz): δ 193.5, 159.5, 153.4, 141.9, 141.6, 140.5, 136.7, 133.2, 131.9, 126.9, 123.7, 123.7, 105.6, 104.4 (2C), 61.0, 56.2 (2C), 55.6, 29.8 ppm. MS: m/z 366 (M⁺, 100%), 351 (44%), 335 (46%), 165 (24%), 89 (26%), 82 (24%). CHN analysis: calculated for C₂₂H₂₂O₅: C, 72.12%, H, 6.05%. found: C, 71.85%, H, 6.04%.

### (2E,4E/Z)-4-(4-Methoxyphenyl)-5-phenyl-1-(3,4,5-trimethoxyphenyl)-penta-2.4-dien-1-one (37)

Compound 37 was synthesized from 2.5 mmol (0.525 g) 3,4,5-trimethoxyacetophenone and 2.5 mmol (0.583 g) 2-(4-mehtoxyphonyl)-3-phenyl-propenal. Yield: 0.75 g (25.0%). Mp: 82-85° C. ¹H-NMR (CDCl₃, 200 MHz): δ 7.80 (AB system, J_{AB}=15.0 Hz, 1H), 7.50-6.90 (m, 12H), 6.57 (AB system, J_{AB}=15D Hz, 1H), 3.90 (s, 3H), 3.88 (s, 3H), 3.87 (s, 6H) ppm. ¹³C-NMR (CDCl₃, 50 MHz): δ 189.6, 159.3, 153.0 (2C),150.0, 140.0, 139.8 (2C), 135.9, 133.7, 130.5 (2C), 130.1 (2C), 129.2, 128.3, 128.2 (2C), 124.1, 114.6 (20), 106.0 (2C), 60.9, 56.3 (2C), 55.3 ppm. MS: m/z 430 (M⁺, 22%), 353 (10%), 235 (18%), 220 (9%), 195 (100%), 135 (32%), 77 (20%). CHN analysis: calculated for C₂₇H₂₆O₅: C, 75.33%, H, 6.09%. found: C, 75.02%, H, 6.11%.

### Example 2

### Cell Proliferation Assay Using Crystal Violet

For quantification, cells are treated with crystal violet, a dye that stains proteins. After washing away of excess dye, the remaining crystal violet is dissolved in a sodium citrate solution. The solution is analyzed photometrically at 590 nm. The optical density of this solution depends on the concentration of crystal violet and therefore on the amount of cells. Appropriate cell density for seeding and duration of treatment with the compounds were determined in preliminary experiments. To reduce effects of evaporation, only the inner 60 wells of 96-well plates (coated with 0.1% gelatine solution for studies with HUVEC) were used for experiments.

Cells were seeded at a density of 5 000 cells per well in 100 μl growth medium (HUVEC) or 20 000 (HCT116), 15 000 (SW480 and 518A2) or 10 000 (HT29) cells per well in 200 μl growth medium in 55 wells per plate. 5 wells were filled with medium only. Additionally, a 96-well plate with cells in 12 wells and medium in 6 wells was prepared as T0-plate. The next day, additional 98 μl of growth medium were added in every well (only HUVEC).

Compounds to be tested were diluted with medium from a 10 mM stock solution in DMSO to a working solution in 50% DMSO. Cells were treated with 2 μl of the working solution 24 h after seeding. Final compound concentrations in the well were 10 μM for the first round of screening. The final DMSO concentration was 0.5%. All treatments and controls were performed in at least triplicates. Cells were incubated at 37° C. and 5% CO₂ for 24 h (HCT116, HT29 and SW480) or 48 h (HUVEC and 518A2). After finishing the addition of compounds, the T0-plate was stained with crystal violet for giving the cell density at time point zero. Before staining, the growth medium was poured away. Wells were filled with 50 μl crystal violet solution for 10 min. Then the staining solution was washed away with water. Since crystal violet is environmentally hazardous the waste was collected and disposed in an appropriate manner. After air drying of the plates the crystal violet on the cells was dissolved in 100 μl of sodium citrate solution, and the absorption was measured with a plate reader at 590 nm.

Mean values were corrected with the absorption of stained medium and the corrected absorption of T0 cells. Cell growth was calculated relative to DMSO control for data comparison. IC₅₀ values (50% inhibition of cell growth) were determined with GraphPad Prism® (Version 4.03) using nonlinear regression (sigmoidal dose-response with variable slope).

Compounds showing an inhibition of proliferation of more than 50% at a concentration of 10 μM were defined as active, and their IC₅₀-values were determined.

In Table 1 shown in the following. IC₅₀-values of compounds according to the invention and reference compounds on HUVEC and HCT116 cell lines, on the colon cancer cell lines SW480 and HT29 and on the melanoma cell line 518A2 are indicated.

TABLE 1
IC₅₀-values in nM of chalcone derivatives according to the invention and reference
compounds on HUVEC and HCT116 cell lines, on the colon cancer cell lines
SW480 and HT29, and on the melanoma cell line 518A2
(data obtained by crystal violet assay; n = 3).

| | IC₅₀ (nM) | | | | |
|---|---|---|---|---|---|
| Compound | HUVEC | HCT116 | SW480 | HT29 | 518A2 |
| 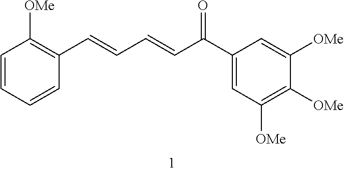 [SMILES: COc1ccccc1/C=C/C=C/C(=O)c1cc(OC)c(OC)c(OC)c1] | 340 | 230 | 520 | 540 | 150 |
| 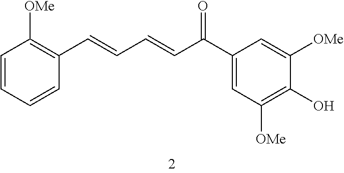 [SMILES: COc1ccccc1/C=C/C=C/C(=O)c1cc(OC)c(O)c(OC)c1] | — | — | 2 680 | 5 790 | 2 750 |
| 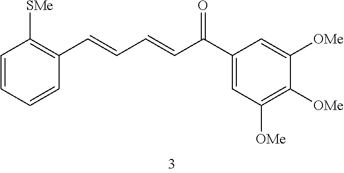 [SMILES: COc1cc(C(=O)/C=C/C=C/c2ccccc2SC)cc(OC)c1OC] | — | — | 260 | 240 | 160 |
| 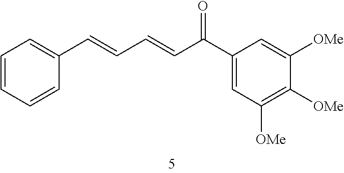 [SMILES: COc1cc(C(=O)/C=C/C=C/c2ccccc2)cc(OC)c1OC] | — | 4 340 | — | — | — |
| 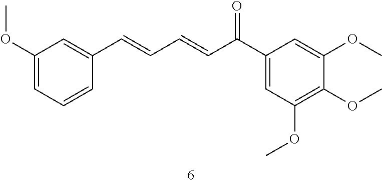 [SMILES: COc1cccc(/C=C/C=C/C(=O)c2cc(OC)c(OC)c(OC)c2)c1] | 3 210 | 4 500 | — | — | — |
| 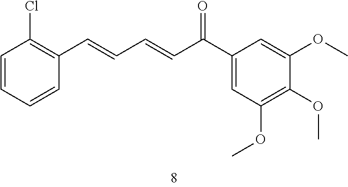 [SMILES: COc1cc(C(=O)/C=C/C=C/c2ccccc2Cl)cc(OC)c1OC] | 1 770 | — | — | — | — |
| 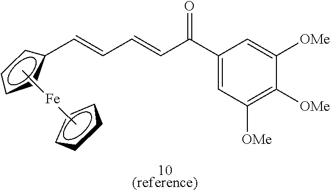 | — | — | >10 000 | >10 000 | >10 000 |
| 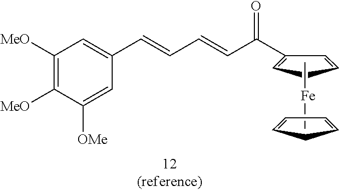 | — | >10 000 | — | — | — |
| 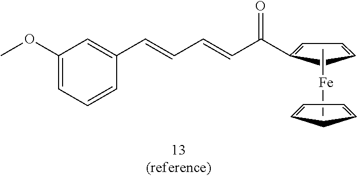 | >10 000 | >10 000 | — | — | — |
| 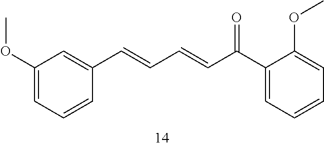 [SMILES: COc1cccc(/C=C/C=C/C(=O)c2ccccc2OC)c1] | 8 810 | >10 000 | — | — | — |
| 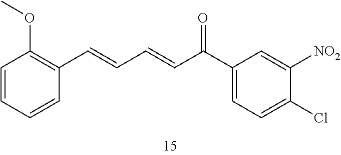 [SMILES: COc1ccccc1/C=C/C=C/C(=O)c1ccc(Cl)c([N+](=O)[O-])c1] | 5 630 | — | — | — | — |
| 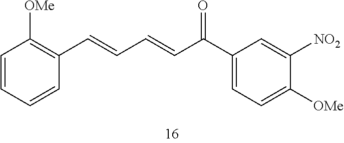 [SMILES: COc1ccccc1/C=C/C=C/C(=O)c1ccc(OC)c([N+](=O)[O-])c1] | — | — | — | — | — |
| 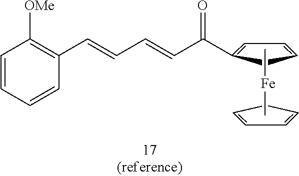 | — | — | — | — | — |
| 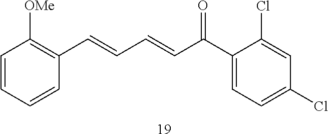 [SMILES: COc1ccccc1/C=C/C=C/C(=O)c1ccc(Cl)cc1Cl ; "19"] | — | — | — | — | — |
| 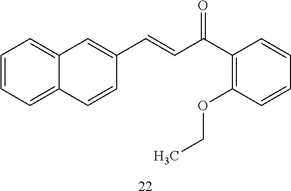 | — | — | 30 | — | 40 |
| 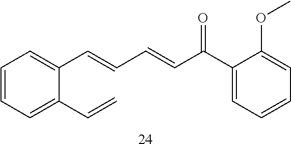 | 760 | 804 | — | — | — |
| 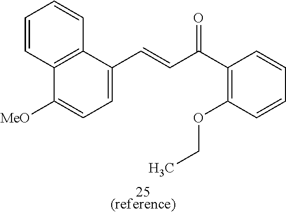 | — | — | >10 000 | — | >10 000 |
| 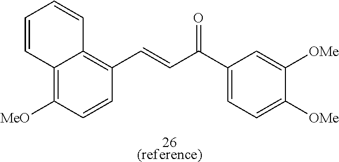 [SMILES: COc1ccc(C(=O)/C=C/c2ccc(OC)c3ccccc23)cc1OC ; "26(reference)"] | >10 000 | >10 000 | — | — | — |
| 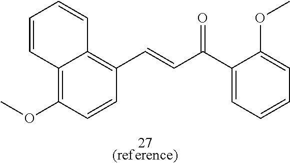 | — | 6 850 | — | — | — |

Lead structure 1 was also tested on the colon carcinoma cell lines HCT116p53 wild type (HCT116p53 wt) and HCT116p53ko as well as on the melanoma cell line SK-MEL 5, on the breast cancer cell line MDA MB231, on human umbilical vein endothelial cells (HUVEC) and vascular smooth muscle cells (VSMC).

The corresponding IC₅₀-values of compound 1 on the above cancer and non-cancer cell lines are indicated in Table 2 shown in the following.

TABLE 2
IC₅₀-values in nM of compound 1 on different cancer and
non-cancer cell lines (crystal violet assay; n = 3).

| Cell line | IC₅₀ (nM) |
|---|---|
| HCT116p53wt | 280 |
| HCT116p53ko | 100 |
| SK-MEL 5 | 150 |
| MDA MB231 | 630 |
| HUVEC | 280 |
| VSMC | >100 000 |

### Example 3

### Cell Viability Assay Using EZ4U Viability Kit (Biomedica)

For quantification, cells are treated with a tetrazolium salt as indicator of cell viability. The method was based on the finding that living cells are able to reduce a slight coloured tetrazolium salt into an intensely coloured formazan derivative. After adding the substrate the cells are incubated for 2 to 4 hours. Then, the solution is analyzed photometrically at 450 nm with 620 nm as reference. Appropriate cell density for seeding and duration of treatment with the substrate were determined in preliminary experiments.

First, cells were seeded in 96 well plates and then growth medium was added to each well to get a final volume of 200 μl. Compounds to be tested were diluted with medium from a 10 mM stock solution in DMSO to get different concentrations and cells were treated with 2 μl of these solutions. The final DMSO concentration was 0.5%. All treatments and controls were performed in at least triplicates. Cells were incubated at 37° C. and 5% CO₂ for 24 h and 48 h. Then, wells were filled with 20 μl substrate solution for 2 to 4 hours and analyzed.

Cell viability was calculated relative to DMSO control for data comparison. IC₅₀ values (50% inhibition of cell growth) were determined with GraphPad Prism® (Version 4.03) using nonlinear regression (sigmoidal dose-response with variable slope).

The compounds 1 and 22 according to the invention were chosen and their activity in different hematological malignancies was investigated. These compounds had shown a highly cytotoxic activity in previous experiments. Table 3 gives an overview of the hematological cell lines used in preliminary experiments.

TABLE 3
Overview of hematological cell lines used in first experiments.

| Cell line | Disease |
|---|---|
| SU-DHL-6 | Non Hodgkin's lymphoma |
| SU-DHL-9 | Non Hodgkin's lymphoma |
| Jurkat | T-cell acute lymphoblastic leukemia |
| CCRF-CEM | T-cell acute lymphoblastic leukemia |
| HL60 | Acute promyelocytic leukemia |
| K562 | Chronic myeloid leukemia |

The IC₅₀-values after 24 and 48 hours in the hematological cell lines used for in vitro experiments are indicated in Table 4 shown in the following.

TABLE 4
IC₅₀-values in nM of the chalcone derivatives 1 and
22 on cell lines representing different hematological malignancies.
Cells were incubated with the compounds for 24 and 48 hours.
IC₅₀-values were calculated of three or more independent
experiments which were carried out in triplicates using the
EZ4U viability kit (Biomedica).

| | IC₅₀ (nM) | | | | | | | | | | | |
|---|---|---|---|---|---|---|---|---|---|---|---|---|
| | SU- | | SU- | | | | CCRF- | | | | | |
| | DHL-6 | | DHL-9 | | Jurkat | | CEM | | HL60 | | K562 | |
| Compound | 24 h | 48 h | 24 h | 48 h | 24 h | 48 h | 24 h | 48 h | 24 h | 48 h | 24 h | 48 h |
| 1 | 180 | 20 | 60 | 50 | 1 110 | 630 | 70 | 70 | 490 | 170 | 20 930 | 50 |
| 22 | 10 | 40 | 30 | 50 | 7 840 | 7 210 | 40 | 40 | 10 | 20 | 17 720 | 13 670 |

### Example 4

### Annexin V-FITC Staining

Annexin V binds to phosphatidylserine which is exposed at the cell surface during execution of apoptosis. First, cells were seeded out at a density of 1.5 to 2.5 million cells per well in 12 well plates and then growth medium was added to each well to get a final volume of 2.5 ml. Compounds to be tested were diluted with medium from a 10 mM stock solution in DMSO to get different concentrations and cells were treated with 25 μl of these solutions. The final DMSO concentration was 0.5%. All treatments and controls were performed twice. Cells were incubated at 37° C. and 5% CO₂ for 24 h and 48 h. Then, growth medium was removed by centrifugation, the supernatant discarded and 200 μl of an Annexin V solution were added. After 10 minutes in the dark, cells were washed once with Annexin buffer and the solution removed by centrifugation. The supernatant was discarded again and cells were suspended in 200 μl of a propidium iodide solution. Cells were measured by flow cytometry with a total of 10 000 events. Data were analyzed with CellQuest Pro™.

Preliminary experiments show that the compounds induce apoptosis in a concentration dependent manner. As shown in FIG. 2, the compounds 1 and 22 induce early apoptosis indicating that the compounds influence cell cycle progression.

Compounds 1 and 22 were also tested on the CLL cell lines MEC-1 and MEC-2 in comparison with fludarabine and cyclophosphamide, two compounds used in the treatment of hematological malignancies (MEC-1 is a fludarabine-resistant cell line).

As shown in FIG. 4, compounds 1 and 22 both induced apoptosis in a lower concentration range than fludarabine and cyclophosphamide after 24 and 48 hours incubation (the value of 40% apoptosis induction which was determined for cyclophosphamide at a concentration of 500 nM, see FIG. 4b, is an outliner since this effect is only seen after 48 hours and not after 24 hours). Accordingly, the compounds of the invention, including compounds 1 and 22, are particularly effective in the medical intervention of hematological malignancies.

### Example 5

### Colony Forming Assay

In a preliminary experiment the impact of chalcone derivatives according to the invention on normal human hematopoietic progenitor cells was assessed. Compounds were added to a standard colony forming assay at concentrations of 1 μM, 0.1 μM, and 0.01 μM (14 days incubation). Compound 1 and compound 22 completely inhibited growth of normal CFU-GM, BFU-E and CFU-GEMM at 1 μM, but had no effect on the 0.1 and 0.01 μM dose levels, as shown in FIG. 3. Interestingly, bystander cells (macrophages, monocytes, lymphocytes) between the colonies remained vital and unaffected in all experiments. The IC₅₀-values obtained by the viability assay in cell lines are in the range of 0.01 μM for compound 1 and 22 after 48 hours in most of the cell lines, as can be seen in Table 4 above. Thus, these data suggest that the compounds have a favorable therapeutic range with preferential killing of malignant cells.

### Example 6

### Cell Cycle Analysis by Using Flow Cytometry

To quantify the cellular DNA content, DNA is visualized by staining with propidium iodide (PI), a fluorescent dye which binds to double stranded DNA. To allow PI to enter cells they are permeabilized with Triton-X. The fluorescence intensity per cell, which correlates with its DNA content, is measured by flow cytometry. The G₀/G₁ peak represents cells in the usual diploid state. Apoptotic cells are characterized through a peak in the FACS histogram below the G₀/G₁ peak (sub G₁) (Nicoletti I et al. J. Immunol. Methods 1991, 139 (2), 271-279):

Since cells in different phases of the cell cycle have a different DNA content this method can also be used for measuring the amount of cells in different stages of the cell cycle. Cells were seeded in 12-well plates at a density 10 fold more than used in 96-well plates in 2 ml growth media. Cells were incubated for one day, until they reached 50-70% confluence. Then they were stimulated with different concentrations of the test compounds, according to the data available from proliferation assays. After incubation at 37° C. for 24 or 48 hours (depending on the cell line), the cells were washed with PBS and detached from the wells with 0.5 ml Trypsin/EDTA. Trypsin was inactivated using 1 ml PBS with 10% FBS. Cells were centrifuged (1 200 rpm, 4 min, 4° C.), washed with PBS and resuspended in 250 μl PI solution containing 0.1% Triton X and stored in the dark at 4° C. for 1 to 5 days.

Cells were measured by flow cytometry 50-150 events/second with a total of 10 000 events using channel FL2-A. Data were analyzed with CellQuest Pro™. Mean values from different repeats were analyzed using GraphPad Prism® 4.03.

It was found that the compounds of the invention influence cell cycle progression, as shown in FIG. 5 for compound 22. The cell cycle analysis indicates that the cytotoxic properties of the compounds may be mediated by the inhibition of cell division (increase of the “DNA>4N” portion in FIG. 5 for cells treated with compound 22 as compared to the control) and the induction of apoptosis (increase of the “Sub G1” portion in FIG. 5 for cells treated with compound 22 as compared to the control).

### Example 7

### In Vitro Drug Test in Primary Chronic Lymphocytic Leukemia (CLL) Cells

In order to evaluate the efficacy of the compounds according to the invention on primary chronic lymphocytic leukemia (CLL) cells, an in vitro screen was carried out using compounds 1 and 22 as well as fludarabine and cyclophosphamide, which are standard therapeutic drugs in CLL, as controls. Cell viability was tested using the CellTiter-Blue Cell Viability Assay (Promega, Madison, Wis., USA) as described in the following.

For quantification, cells were treated with resazurin (7-hydroxy-3H-phenoxazin-3-one 10-oxide) as indicator of cell viability. The method is based on the finding that living cells are able to reduce the non-fluorescent blue dye into the pink colored and highly red fluorescent resorufine. After adding the substrate the cells are incubated for 2 to 4 hours. Then, the solution is analyzed at 560 nm _{Ex}/590 nm _{Em} on a fluorometer. Appropriate cell density for seeding and duration of treatment with the substrate were determined in preliminary experiments.

First, cells were seeded in 96 well plates and then growth medium was added to each well to get a final volume of 80 μl. Compounds to be tested were diluted with medium from a 10 mM stock solution in DMSO to get different concentrations, and 20 μl of these solutions were added to the wells. The final DMSO concentration was 0.5%. All treatments and controls were performed in at least triplicates. Cells were incubated at 37° C. and 5% CO₂ for 48 h. Then, 20 μl substrate solution was added to the wells, plates were incubated for 3 hours and analyzed.

The effect of compounds 1 and 22 according to the invention as well as the control drugs fludarabine and cyclophosphamide on the viability of the primary CLL cells is shown in FIG. 6. As can be seen, compounds 1 and 22 are effective in CLL cells at lower concentrations than the standard therapeutic drugs fludarabine and cyclophosphamide. These results demonstrate that the compounds of the present invention are particularly effective in the treatment of cancer, including malignant hematological diseases/disorders such as chronic lymphocytic leukemia.

### Example 8

### In Vitro Drug Test in a Co-Culture Model of Prima Chronic Lymphocytic Leukemia (CLL) Cells and Stromal Cells

In many tumors, the microenvironment plays a major role for the survival of malignant cells. In chronic lymphocytic leukemia (CLL), it has been shown that interaction with the microenvironment, among other things, promotes the survival of tumor cells during chemotherapy. Thus, co-culture models have been developed which better reflect the in vivo situation. The inventors used such a pre-clinical model for drug incubation to study the effect of stromal cells on viability. Cell viability was tested using the CellTiter-Blue Cell Viability Assay (Promega, Madison, Wis., USA) as described in Example 7.

As shown in FIG. 7, the presence of stromal cells reduces the effect of all tested compounds on the tumor cells, thus supporting better survival. However, compounds 1 and 22 according to the invention still showed higher cytotoxicity than the control drugs fludarabine and cyclophosphamide. These findings further confirm the utility of the compounds of the invention in the medical intervention of cancer, including particularly chronic lymphocytic leukemia.

### Example 9

### Effect on Healthy Peripheral Blood Mononuclear Cells (PBMC)

The impact of the compounds according to the invention on healthy cells was assessed using peripheral blood mononuclear cells (PBMC). Cell viability was tested using the CellTiter-Blue Cell Viability Assay (Promega, Madison, Wis., USA) as described in Example 7.

The results are shown in FIG. 8 and conform with previous experiments on hematopoietic stem cells. In comparison to CLL cells, healthy PBMC were much less affected by the drugs tested, including compounds 1 and 22 according to the invention. As also shown in FIG. 8, fludarabine was the most toxic of the tested compounds, the two tested chalcones 1 and 22 were less toxic, and cyclophosphamide did not influence viability of healthy PBMC under the conditions tested. These data indicate that the compounds of the present invention are less toxic in healthy peripheral blood mononuclear cells than, e.g., the standard therapeutic drug fludarabine. Accordingly, the compounds provided herein are particularly suitable as pharmaceutical agents.

1. A compound of formula (I) 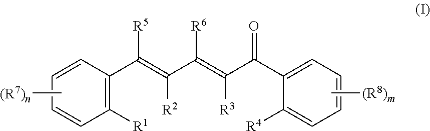
wherein:
R¹ and R² are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl), or R¹ and R² are mutually linked to form a group —C(R⁹)═C(R⁹)—;
R³ and R⁴ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl), or R³ and R⁴ are mutually linked to form a group —CH(R⁹)— or —CH(R⁹)—CH(R⁹)—;
R⁵ and R⁶ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl);
each R⁷ is independently selected from halogen, —NO₂, —CF₃, —ON, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl); wherein, if n is equal to or greater than 2, two groups R⁷ which are attached to adjacent carbon atoms may be mutually linked to form a group —C(R⁹)═C(R⁹)—C(R⁹)═C(R⁹)— or a group —O—CH(R⁹)—O—;
each R⁸ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl); wherein, if m is equal to or greater than 2, two groups R⁸ which are attached to adjacent carbon atoms may be mutually linked to form a group —C(R⁹)═C(R⁹)—C(R⁹)═C(R⁹)— or a group —O—CH(R⁹)—O—;
each R⁹ is independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl);
n is 0, 1, 2, 3, or 4; and
m is 0, 1, 2, 3, or 4;
or a pharmaceutically acceptable salt, solvate or prodrug thereof for use in the treatment or prevention of cancer.

2. The compound of claim 1, wherein R¹ is selected from hydrogen, halogen, —NO₂, —OH, —O(C₁₋₄ alkyl), —SH, or —S(C₁₋₄ alkyl), and R² is hydrogen.

3. The compound of claim 1, wherein R¹ and R² are mutually linked to form a group —CH═CH—.

4. The compound of any of claims 1 to 3, wherein R³ is hydrogen, and R⁴ is selected from hydrogen, halogen, —NO₂, —OH, —O(C₁₋₄ alkyl), —SH, or —S(C₁₋₄ alkyl).

5. The compound of any of claims 1 to 3, wherein R³ and R⁴ are mutually linked to form a group —CH₂— or —CH₂—CH₂—.

6. The compound of any of claims 1 to 5, wherein R⁵ and R⁶ are each hydrogen.

7. The compound of any of claims 1 to 6, wherein each R⁷ is independently selected from halogen, —NO₂, —OH, —O(C₁₋₄ alkyl), —SH, or —S(C₁₋₄ alkyl).

8. The compound of any of claims 1 to 7, wherein each R⁸ is independently selected from halogen, —NO₂, —OH, —O(C₁₋₄ alkyl), —SH, or —S(C₁₋₄ alkyl).

9. The compound of any of claims 1 to 8, wherein n is 1.

10. The compound of any of claims 1 to 9, wherein m is 1 or 3.

11. The compound of claim 1, wherein said compound is a compound of one of the following formulae 1 or 22: 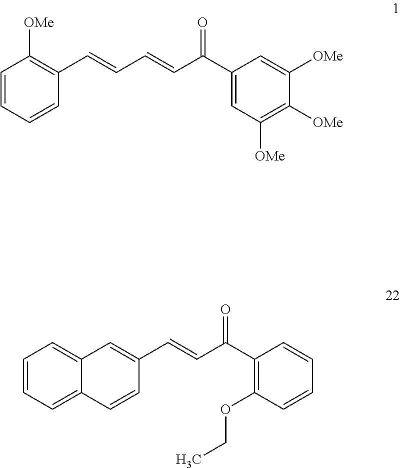 [SMILES: COc1ccccc1/C=C/C=C/C(=O)c1cc(OC)c(OC)c(OC)c1 ; CCOc1ccccc1C(=O)/C=C/c1ccc2ccccc2c1]
or a pharmaceutically acceptable salt, solvate or prodrug thereof
for use in the treatment or prevention of cancer.

12. A pharmaceutical composition comprising the compound of any of claims 1 to 11 and a pharmaceutically acceptable excipient for use in the treatment or prevention of cancer.

13. A method of treating or preventing cancer, the method comprising the administration of the compound of any of claims 1 to 11 or the pharmaceutical composition of claim 12 to a subject in need of such a treatment or prevention.

14. The compound of any of claims 1 to 11 or the pharmaceutical composition of claim 12 or the method of claim 13, wherein the cancer is a malignant hematological disease/disorder.

15. The compound of claim 14 or the pharmaceutical composition of claim 14 or the method of claim 14, wherein the malignant hematological disease/disorder is selected from Hodgkin's disease, non-Hodgkin's lymphoma. Burkitt's tumor, peripheral or cutaneous T-cell lymphoma, mycosis fungoides, Sézary's disease, T-zone lymphoma, lymphoepithelioid lymphoma. Lennert's lymphoma, peripheral T-cell lymphoma, lymphosarcoma, a malignant immunoproliferative disease. Waidenström's macroglobulinaemia, alpha heavy chain disease, gamma heavy chain disease. Franklin's disease, an immunoproliferative small intestinal disease, Mediterranean disease, multiple myeloma, Kahler's disease, myelomatosis, plasma cell leukaemia, lymphoid leukaemia, acute lymphoblastic leukaemia, chronic lymphocytic leukaemia, subacute lymphocytic leukaemia, prolymphocytic leukaemia, hairy-cell leukaemia, leukaemic reticuloendotheliosis, adult T-cell leukaemia, myeloid leukaemia, acute myeloid leukaemia, chronic myeloid leukaemia, subacute myeloid leukaemia, myeloid sarcoma, chloroma, granulocytic sarcoma, acute promyelocytic leukaemia, acute myelomonocytic leukaemia, chronic BCR-ABL negative myeloproliferative disorders, polycythaemia vera, essential thrombocythemia, idiopathic myelofibrosis, monocytic leukaemia, acute erythraemia or erythroleukaemia, acute erythraemic myelosis, Di Guglielmo's disease, chronic erythraemia, Heilmeyer-Schöner disease, acute megakaryoblastic leukaemia, mast cell leukaemia, acute panmyelosis, acute myelofibrosis, or Letterer-Siwe disease.

16. The compound of any of claims 1 to 11 or the pharmaceutical composition of claim 12 or the method of claim 13, wherein the cancer is selected from breast cancer, genitourinary cancer, prostate tumor, hormone-refractory prostate tumor, lung cancer, small cell lung tumor, non-small cell lung tumor, gastrointestinal cancer, hepatocellular carcinoma, colorectal tumor, colon cancer, gastric cancer, epidermoid cancer, epidermoid head and/or neck tumor, mouth tumor, melanoma, ovarian cancer, pancreas cancer, neuroblastoma, bladder cancer, renal cancer, or brain cancer.

17. The pharmaceutical composition of any of claims 12 or 14 to 16 or the compound of any of claims 1 to 11 or 14 to 16 or the method of any of claims 13 to 16, whereby the pharmaceutical composition or the compound is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route.

18. The compound of any of claims 1 to 11 or 14 to 17 or the pharmaceutical composition of any of claims 12 or 14 to 17 or the method of any of claims 13 to 17, whereby the compound or the pharmaceutical composition is to be administered in combination with an anti-proliferative drug, an anticancer drug, a cytostatic drug, a cytotoxic drug and/or radiotherapy.

19. The method of any of claims 13 to 18, wherein the subject is a human.

20. A compound of formula (Ia) 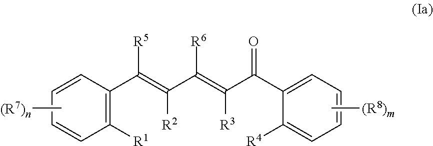
wherein:
R¹ and R² are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl), or R¹ and R² are mutually linked to form a group
R³ and R⁴ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —ON, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl), or R³ and R⁴ are mutually linked to form a group —CH(R⁹)— or —CH(R⁹)—CH(R⁹)—;
R⁵ and R⁶ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl);
each R⁷ is independently selected from halogen, —NO₂, —CF₃, —CN, O alkyl, —OH, —O(C alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl); wherein, if n is equal to or greater than 2, two groups R⁷ which are attached to adjacent carbon atoms may be mutually linked to form a group —C(R⁹)═C(R⁹)—C(R⁹)═C(R⁹)— or a group —O—CH(R⁹)—O—;
each R⁸ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —C—O—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, O alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl); wherein, if m is equal to or greater than 2, two groups R⁸ which are attached to adjacent carbon atoms may be mutually linked to form a group —C(R⁹)═C(R⁹)—C(R⁹)═C(R⁹)— or a group —O—CH(R⁹)—O—;
each R⁹ is independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl);
n is 0, 1, 2, 3, or 4; and
m is 0, 1, 2, 3, or 4;
and further wherein R¹ and R² are mutually linked to form a group —C(R⁹)═C(R⁹)—, and/or R³ and R⁴ are mutually linked to form a group —CH(R⁹)— or —CH(R⁹)—CH(R⁹)—;
or a pharmaceutically acceptable salt, solvate or prodrug thereof.

21. The compound of claim 20, wherein said compound is a compound of formula (IIa) 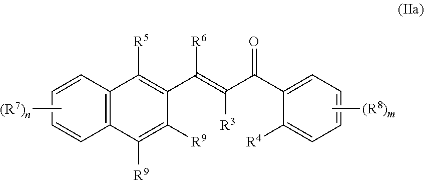
wherein:
R³ and R⁴ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl), or R³ and R⁴ are mutually linked to form a group —CH(R⁹)— or —CH(R⁹)—CH(R⁹)—;
R⁵ and R⁶ are each independently selected from hydrogen, halogen, —NO₂, —CF₃, —CN, C₁₋₄ alkyl, —OH, —O(C₁₋₄ alkyl), —SH, —S(C₁₋₄. alkyl), —NH₂, —NH(C₁₋₄ alkyl), —N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl);
each R⁷ is independently selected from halogen, —NO₂, —CF₃, —CN, C₁₋₄ alkyl, —OH, —O(C alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —ON, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl); wherein, if n is equal to or greater than 2, two groups R⁷ which are attached to adjacent carbon atoms may be mutually linked to form a group —C(R⁹)═C(R⁹)—C(R⁹)═C(R⁹)— or a group —O—CH(R⁹)—O—;
each R⁸ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl); wherein, if m is equal to or greater than 2, two groups R⁵ which are attached to adjacent carbon atoms may be mutually linked to form a group —C(R⁹)═C(R⁹)—C(R⁹)═C(R⁹)— or a group —O—CH(R⁹)—O—;
each R⁹ is independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), alkyl)(C₁₋₄ alkyl), —SO₂—NH₂, —SO₂—NH(C₁₋₄ alkyl), or —SO₂—N(C₁₋₄ alkyl)(C₁₋₄ alkyl);
n is 0, 1, 2, 3, or 4; and
m is 0, 1, 2, 3, or 4;
or a pharmaceutically acceptable salt, solvate or prodrug thereof.

22. The compound of claim 21, wherein said compound is a compound of the following formula 22: 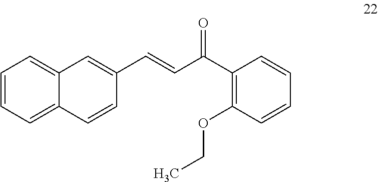 [SMILES: CCOc1ccccc1C(=O)/C=C/c1ccc2ccccc2c1]
or a pharmaceutically acceptable salt, solvate or prodrug thereof.

23. The compound of claim 20, wherein said compound is a compound of formula (IIIa) 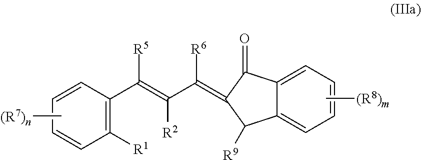
wherein:
R¹ and R² are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —ON, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl), or R¹ and R² are mutually linked to form a group —C(R⁹)═C(R⁹)—;
R⁵ and R⁶ are each independently selected from hydrogen, halogen, —NO₂, —CF₃, —ON, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl);
each R⁷ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl); wherein, if n is equal to or greater than 2, two groups R⁷ which are attached to adjacent carbon atoms may be mutually linked to form a group —C(R⁹)═C(R⁹)—C(R⁹)═C(R⁹)— or a group —O—CH(R⁹)—O—;
each R⁸ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), alkyl)(C₁₋₄ alkyl), —SO₂—NH₂, —SO₂—NH(C₁₋₄ alkyl), or —SO₂—N(C₁₋₄ alkyl)(C₁₋₄ alkyl); wherein, if m is equal to or greater than 2, two groups R⁸ which are attached to adjacent carbon atoms may be mutually linked to form a group —C(R⁹)═C(R⁹)—C(R⁹)═C(R⁹)— or a group —O—CH(R⁹)—O—;
each R⁹ is independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl);
n is 0, 1, 2, 3, or 4; and
m is 0, 1, 2, 3, or 4;
or a pharmaceutically acceptable salt, solvate or prodrug thereof.

24. The compound of claim 20, wherein said compound is a compound of formula (IVa) 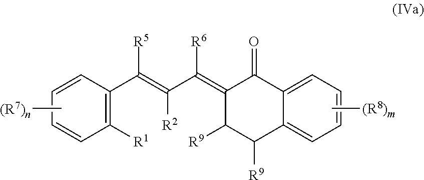
wherein:
R¹ and R² are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl), or R¹ and R² are mutually linked to form a group —C(R⁹)═C(R⁹)—;
R⁵ and R⁶ are each independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂— (C₁₋₄ alkyl)(C₁₋₄ alkyl);
each R⁷ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl) (C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl); wherein, if n is equal to or greater than 2, two groups R⁷ which are attached to adjacent carbon atoms may be mutually linked to form a group —C(R⁹)═C(R⁹)—C(R⁹)═C(R⁹)— or a group —O—CH(R⁹)—O—;
each R⁸ is independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl); wherein, if m is equal to or greater than 2, two groups R⁸ which are attached to adjacent carbon atoms may be mutually linked to form a group —C(R⁹)═C(R⁹)—C(R⁹)═C(R⁹)— or a group —O—CH(R⁹)—O—;
each R⁹ is independently selected from hydrogen, halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), —SO₂—N(C₁₋₄ alkyl)(C₁₋₄ alkyl), or phenyl optionally substituted with one or more groups independently selected from halogen, —NO₂, —CF₃, —CN, C₁₋₄ alkyl, —OH, —O(C₁₋₄ alkyl), —SH, —S(C₁₋₄ alkyl), —NH₂, —NH(C₁₋₄ alkyl), —N(C₁₋₄ alkyl)(C₁₋₄ alkyl), —CO—NH₂, —CO—NH(C₁₋₄ alkyl), —CO—N(C₁₋₄ alkyl)(C₁₋₄ alkyl), —SO₂—NH₂, —SO₂—NH(C₁₋₄ alkyl), or —SO₂—N(C₁₋₄ alkyl)(C₁₋₄ alkyl);
n is 0, 1, 2, 3, or 4; and
m is 0, 1, 2, 3, or 4;
or a pharmaceutically acceptable salt, solvate or prodrug thereof.

25. A pharmaceutical composition comprising the compound of any of claims 20 to 24 and a pharmaceutically acceptable excipient.

26. The compound of any of claims 20 to 24 or the pharmaceutical composition of claim 25 for use as a medicament.

27. A method of treating or preventing a disease or disorder, the method comprising the administration of the compound of any of claims 20 to 24 or the pharmaceutical composition of claim 25 to a subject in need of such a treatment or prevention.

28. The method of claim 27, wherein the subject is a human.
